(19) United States
(12) Patent Application Publication
(10) Pub. No.: US 2014/0030709 A1
(43) Pub. Date: Jan. 30, 2014

(54) Biomarker for Detecting High-Altitude Adaptation and High-Altitude Pulmonary Edema

(71) Applicants: Bhavana Prasher, New Delhi (IN); Shilpi Aggarwal, New Delhi (IN); Mohammed Abdul Qadar Pasha, New Delhi (IN); Mitali Mukerji, New Delhi (IN)

(72) Inventors: Bhavana Prasher, New Delhi (IN); Shilpi Aggarwal, New Delhi (IN); Mohammed Abdul Qadar Pasha, New Delhi (IN); Mitali Mukerji, New Delhi (IN)

(73) Assignee: COUNCIL OF SCIENTIFIC & INDUSTRIAL RESEARCH, New Delhi (IN)

(21) Appl. No.: 13/703,308

(22) PCT Filed: Oct. 13, 2011
(86) PCT No.: PCT/IB2011/002415

§ 371 Date: Apr. 12, 2013

(30) Foreign Application Priority Data
Dec. 9, 2010 (IN) 1336/DEL/2010

(51) Int. Cl. C12Q 1/68

(52) U.S. Cl. CPC C12Q 1/6883
USPC 435/6.11; 536/23.2; 536/24.33

(57) ABSTRACT

Present invention relates to the Biomarkers for detecting high altitude adaptation and hypoxia responsiveness and the method thereof. The invention specifically relates to the Gene variants SNPIDs rs479200 and rs480902 in the first intron of EGLN1 (Prolyl Hydroxylase 2) gene as biomarkers for adaptation to high altitude and predisposition for high altitude pulmonary edema and hypoxia responsiveness using a novel integrative approach of phenotyping concepts of Ayurveda with population genetics, and disease genomics. More specifically, the C allele of SNP ID rs480902 and T allele of rs479200 of EGLN1 gene is more frequent in patients of HAPE and nearly absent in native highlanders. The present invention also provides primers and methods suitable for the detection of these allelic variants for the prediction of individual's adaptability to high altitude and hypoxia and/or the genetic analysis of the EGLN1 gene in a population.

The following specification particularly describes the invention and the manner in which it is to be performed:

### FIELD OF THE INVENTION

The present invention relates to a biomarker useful for predicting predisposition of an individual to high altitude adaptation and high-altitude pulmonary edema (HAPE) characterized in having single nucleotide polymorphism C/T at position 27 in SEQ ID NO. 1 and T/C at position 27 in SEQ ID NO. 2 of the EGLN1 gene. The invention also relates to the method for detecting high-altitude adaptation and high-altitude pulmonary edema using EGLN-1 related markers. The invention relates to the biomarkers associated with low and high risk to the sickness related to high altitude using a novel integrative approach of phenotyping concepts of Ayurveda with population genetics, and disease genomics. More particularly, the present invention relates to allelic variants of the human Prolyl Hydroxylase 2 gene and provides primers suitable for the detection of these allelic variants for the prediction of individual's adaptability to high altitude and hypoxia and/or the genetic analysis of the EGLN1 gene in a population.

### BACKGROUND OF THE INVENTION

Discovery of biomarkers for disease susceptibility and response to external environment is met with many challenges owing to large amount of inter individual variability within populations (Frazer et al., 2009; McClellan and King, 2010). It is insufficient to describe populations using ethnicity labels since there exists broad Endophenotypes within populations that are differently predisposed to different diseases and environmental conditions. Age, sex and ethnicity matched case control studies are highly dependent upon strong contrasts in disease susceptibility and/or adaptability between cases and controls where controls do not have obvious clinical disease and mostly comprise of heterogeneous endophenotypes. It is being realized that identification of endophenotypes within normal controls corresponding to contrasting disease susceptibility/adaptability is likely to lead to more effective biomarker discovery. Ayurveda, an ancient system of Indian medicine documented and practiced since 1500 B.C. deals with inter-individual variability for personalized and predictive medicine. This system of medicine, phenotypically classifies individuals into seven broad constitution types termed Prakriti, among which Vata (V), Pitta (P) and Kapha (K) are the most contrasting constitution types that exhibit readily recognizable phenotypes (Sharma, P. V. 2000; Prasher et al., 2008). Integration of this endophenotyping approach with genetic studies can bridge the gap of phenotype to genetic variations.

Since presence of such genetic variations might also determine course of disease and lead to development of discrete disease states, detecting their presence can help in development of personalized treatment plan for the same. This would also allow development and dissemination of preventive regimen involving diet and lifestyle in order to avoid or delay the development of such disease conditions.

Difference in genome wide expression and biochemical profiles amongst these contrasting constitutions of Ayurveda endophenotype samples has been demonstrated by Prasher et al 2008. In the differentially expressed set a significant over-representation of hub and housekeeping genes was observed that could lead to system-wide effects. EGLN1 was found to be amongst 251 differentially expressed genes between three contrasting endophenotypes (Vata (V), Pitta(P), Kapha(K)) within normal individuals of Indo-European (IE) origin from an earlier study (Prasher et al 2008).

EGLN1 (prolyl hydroxylase 2) is an oxygen sensor gene that plays a key role in oxygen homeostasis through regulation of HIF-1A, (Fong and Takeda, 2008) the hypoxia inducible factor and therefore is of importance in a large number of cellular, physiological and systemic processes.

In normoxic conditions, EGLN1 hydroxylates the constitutively expressed HIF at two of it's proline residues, leading to its polyubiquitination by the Von Hippel Lindau (VHL) E-3 ligase complex and degradation by the proteosomal machinery (Semenza, 2009). In conditions of hypoxia, EGLN1 is inactive leading to stabilization of HIF that induces the expression of genes which mediate adaptive responses at cellular (through glycolytic enzymes, hemeoxygenase); local (vascular endothelial growth factor) and systemic (erythropoietin) level (Smith et al 2008). Variations in EGLN1 could contribute to differences in physiological response to hypoxia thereby affecting performance in high altitude conditions.

The present invention deals with EGLN1 variations that distinguish constitution types in subjects who develop HAPE, a condition that normally occurs in un-acclimated sojourners at altitudes above 2,500 m, and accounts for most of the deaths due to altitude sickness (Rodway et al., 2003).

The disease is characterised by hypoxia induced pulmonary vasoconstriction caused by endothelial dysfunction and intravascular fluid retention. While some families and individuals are at risk, those with a long ancestry at high altitude have a lower risk. Moreover, individuals who have had HAPE are at a greater risk of repeat events. Such data support a strong genetic component to HAPE susceptibility. It is likely that long term exposure to high altitude provides a natural positive adaptive pressure to alleles that prevent the illness (Ahsan et al., 2004). Rapid descent of HAPE patients not only prevents exacerbation of HAPE but also improves the pathogenesis of the disease (Hackett, et al., 2001).

Genetic Predisposition:

Though variations in genes of pathways related to hypoxia such as HIF-1, endothelial function (ET1) and vascular remodeling (ACE, eNOS) have been studied (Mortimer et al., 2004; Qadar Pasha et al., 2001; Morrell et al., 1999; Smith et al., 2008; Liu et al., 2007; Ahsan et al., 2004) none of the studies so far have reported a involvement of EGLN1 in HAPE. These studies have been carried out as case control association studies.

Recently a genome wide population study carried out on Tibetans has reported a haplotype in EGLN1 and PPARA gene to be associated with HAA (Tatum et al 2010). However, this does not specifically demarcate the SNP/region of DNA of the present invention neither have they studied HAPE or frequency of the haplotype in the populations residing at high altitude globally. None of the genetic predisposition studies have looked at endophenotypes in the populations who might be differently predisposed to high altitude adaptation (HAA).

### DEFINITIONS

SNP—Single nucleotide polymorphisms (SNP) are the most common type of genetic variation. A SNP is a single base pair mutation at a specific locus, usually consisting of two alleles.

Predictive marker—A genetic variation (e.g. SNP) that is associated with risk or protection to a particular disease or response to therapy

Prakriti or constitution type—Prakriti or body constitution of an individual is a consequence of the relative proportion of three entities (Tri-Doshas), Vata (V), Pitta (P) and Kapha (K). The Tri-doshas work in conjunction and maintain homeostasis throughout the lifetime starting from fertilization. Distinct properties and functions have been ascribed to each Dosha

Vata—Vata is one of the three entities comprising Tridoshas. It contributes to manifestation of shape, cell division, signaling, movement, excretion of wastes, cognition and also regulates the activities of Kapha and Pitta.

Kapha—Kapha is one of the three entities comprising Tridoshas. It is responsible for growth and maintenance of structure, storage and stability.

Pitta—Pitta is one of the three entities comprising Tridoshas. It is primarily responsible for metabolism, thermo-regulation, energy homeostasis, pigmentation, vision, and host surveillance.

Endophenotypes—Individuals within a population who can be classified based on similarities of physical, physiological, behavioral characteristics etc.

Hypoxia—Hypoxia is a pathological condition in which the body as a whole (generalized hypoxia) or a region of the body (tissue hypoxia) is deprived of adequate oxygen supply.

HGDP—Human Genome Diversity panel

CEPH—Centre d'Etude du Polymorphisme Humain

IGVC—Indian Genome Variation Consortium

IE pool—Pool of Indo-European large populations.

### OBJECTIVE OF THE INVENTION

The main object of the present invention is to provide a biomarker useful for predicting predisposition of an individual to high altitude adaptation and high-altitude pulmonary edema (HAPE) characterized in having single nucleotide polymorphism C/T at position 27 in SEQ ID NO. 1 (rs479200) and T/C at position 27 in SEQ ID NO. 2 (rs480902) of the EGLN1 gene.

### SUMMARY OF THE INVENTION

Accordingly, the present invention provides biomarkers for detecting high altitude adaptation and hypoxia responsiveness and the method thereof.

In an embodiment to the present invention, wherein the biomarkers are the gene variants of EGLN1 gene having single nucleotide polymorphism C/T at position 27 in SEQ ID NO. 1 (rs479200) and T/C at position 27 in SEQ ID NO. 2 (rs480902) of the EGLN1 gene useful for predicting predisposition of an individual to high altitude adaptation and high-altitude pulmonary edema (HAPE).

In another embodiment to the present invention, the frequency of occurrence of ‘T’ allele of SNPID rs479200 in HAPE patients is 0.64 and Kapha Prakriti individuals is 0.71.

In still another embodiment to the present invention, the frequency of occurrence of ‘T’ allele of rs479200 in native highlanders is 0.21 and Pitta Prakriti individuals is 0.36.

In yet another embodiment to the present invention the frequency of occurrence of ‘T’ allele of rs479200 in HAPE patients is 0.64 and native highlanders is 0.21 (p value 4.36×10⁻¹⁷).

In still another embodiment of the present invention the frequency of occurrence of ‘T’ allele of rs479200 in HAPE patients is 0.64 and Pitta Prakriti individuals is 0.36 (p value 0.000272).

In further embodiment to the present invention the frequency of occurrence of ‘T’ allele of rs479200 in Kapha Prakriti individuals is 0.71 and Pitta Prakriti individuals is 0.36 (p value 2.17×10⁻⁴).

In another embodiment to the present invention ‘TT’ genotype of SNP ID rs479200 of EGLN1 gene is more frequent in Kapha Prakriti and is associated with high risk to HAPE.

In still another embodiment to the present invention ‘TT’ genotype of rs479200 was over-represented in the Kapha Prakriti types and was also found to correlate with higher expression of EGLN1 gene

In yet another embodiment to the present invention ‘TC and CC’ genotype of rs479200 that was under-represented in the Kapha Prakriti types and was also found to correlate with lower expression of EGLN1 gene

In another embodiment to the present invention expression of EGLN1 associated with ‘TC and CC’ genotype of rs479200 was significantly different from ‘TT’ genotype (p value=0.017)

In yet another embodiment to the present invention the frequency of occurrence of ‘C’ allele of rs480902 in HAPE patients is 0.63 and Kapha individuals is 0.69.

In further another embodiment to the present invention the frequency of occurrence of ‘C’ allele of SNP ID rs480902 in native high landers is 0.28 and Pitta individuals is 0.36.

In another embodiment to the present invention the frequency of occurrence of ‘C’ allele of rs480902 in HAPE patients is 0.63 and Pitta individuals is 0.36 (p value=0.000447).

In still another embodiment to the present invention the frequency of occurrence of ‘C’ allele of rs480902 in HAPE patients is 0.63 and in native high landers is 0.28 (p value 7.69×10⁻¹²).

In another embodiment to the present invention the frequency of occurrence of ‘C’ allele of rs480902 in Kapha individuals is 0.69 and Pitta individuals is 0.36 (p value 4.55×10⁻⁴).

In another embodiment to the present invention ‘CC’ genotype of SNP ID rs480902 of EGLN1 gene is more frequent is Kapha Prakriti types and is associated with high risk to HAPE.

In further embodiment to the present invention ‘T’ allele of SNP ID rs480902 and ‘C’ allele of rs479200 of EGLN1 gene is associated with low risk to HAPE and is nearly fixed in native highlanders

In further embodiment to the present invention the ‘C’ allele of SNP ID rs480902 and ‘T’ allele of rs479200 of EGLN1 gene is more frequent in HAPE and nearly absent in native highlanders

In yet another embodiment of the present invention the Primer useful for amplifying biomarkers having sequences selected from the group comprising of:
SEQ ID NO. 5 represented by 5′ TATTCTGTCTTCGGCAGAGG 3′ which is a forward primer;
SEQ ID NO. 6 represented by 5′ AGCAAGCAAAGAAAGGCGAG 3′ which is a reverse primer;
SEQ ID NO. 7 represented by 5′ AGGACTTTTATTATTGCTTGTTA 3′ which is a SNaPshot Primer;
SEQ ID NO. 8 represented by 5′ ATTGCTTGGGAGGTTGTTGG 3′ which is a forward primer;
SEQ ID NO. 9 represented by 5′ TTTCACTGGAGTTGTGGGAG 3′ which is a reverse primer;
SEQ ID NO. 10 represented by'5′ GATCTCCCAGTGACTCA 3′ which is a SNaPshot Primer.

In yet another embodiment of the present invention a method of preparing biomarkers wherein the said method comprises:—
a) isolating genomic DNA from human subject;
b) designing and synthesizing forward and reverse oligonucleotide primers having SEQ ID NOs: 8 and 9 for positive strand of intron 1 of the EGLN1 gene;
c) amplifying positive strand of intron 1 of the EGLN1 gene having SEQ ID NO. 3 using primers synthesized in step b to obtain biomarker of sequence ID No. 2 having SNPID rs480902;
d) designing and synthesizing forward and reverse oligonucleotide primers having SEQ ID NOs: 5 and 6 for negative strand of intron 1 of the EGLN1 gene;
e) amplifying negative strand of intron 1 of the EGLN1 gene having SEQ ID NO. 4 using primers synthesized in step d to obtain biomarker of sequence ID No. 1 having SNPID rs479200;
f) determining SNPs in HAPE patients and native highlander subjects using snap shot primers of SEQ ID NOs: 7 and 10 for SNPID rs479200 and SNPID rs480902 respectively;

In yet another embodiment of the present invention a method for detecting high altitude adaptation and predisposition of an individual to high altitude pulmonary edema, wherein the said method comprising the steps of:
a) isolating genomic DNA from human subject;
b) designing and synthesizing forward and reverse oligonucleotide primers having SEQ ID NOs: 8 and 9 for positive strand of intron 1 of the EGLN1 gene;
c) amplifying positive strand of intron 1 of the EGLN1 gene having SEQ ID NO. 3 using primers synthesized in step b to obtain biomarker of sequence ID No. 2 having SNPID rs480902;
d) designing and synthesizing forward and reverse oligonucleotide primers having SEQ ID NOs: 5 and 6 for negative strand of intron 1 of the EGLN1 gene;
e) amplifying negative strand of intron 1 of the EGLN1 gene having SEQ ID NO. 4 using primers synthesized in step d to obtain biomarker of sequence ID No. 1 having SNPID rs479200;
f) determining SNPs in HAPE patients and native highlander subjects using snap shot primers of SEQ ID NOs: 7 and 10 for SNPID rs479200 and SNPID rs480902 respectively,
g) computing the frequencies of TT,TC and CC genotypes in the populations of step (f) for establishing the association of the genotypes with high altitude adaptation and high altitude pulmonary edema;
h) predicting and statistically analyzing differences in the distribution of the allelic variants (T, C) in the population wherein TT genotype of rs479200 and CC genotype of rs480902 in EGLN1 gene are at high risk to high altitude pulmonary edema and CC genotype of rs479200 and TT genotype of rs480902 in EGLN1 gene are at low risk to high altitude pulmonary edema.

In another embodiment of the present invention a kit for detecting high altitude adaptation and predisposition of an individual to high altitude pulmonary edema in human subject, wherein the said kit comprising:
i. primers having SEQ ID NO. 5, 6, 7, 8, 9, 10;
ii. suitable reagents;
iii. instruction manual.

In another aspect of the present invention use of Biomarkers for predicting predisposition of an individual to high altitude adaptation and high-altitude pulmonary edema (HAPE) characterized in having single nucleotide polymorphism T/C at position 21782 in SEQ ID NO. 3 (rs480902) and C/T at position 12964 in SEQ ID NO. 4 (rs479200) of the EGLN1 gene.

In another aspect of the present invention the Biomarkers detect high altitude adaptation and hypoxia responsiveness and the method thereof comprises:
1. Endophenotyping of normal healthy human subjects for analysis of Prakriti types and recruitment of subjects for genomic analysis,
2. Isolation of genomic DNA and RNA followed by genotyping by known methods,
3. Detection of single nucleotide polymorphisms in EGLN1 gene using set of PCR primers for SNPID rs479200 having SEQ ID NOs: 5 and 6.
4. Detection of single nucleotide polymorphisms in EGLN1 gene using set of PCR primers for SNPID rs480902 having SEQ ID NOs: 8 and 9.
5. Determining of SNPs in HAPE patients and native highlander subjects using snap shot primers of SEQ ID Nos: 7 and 10 for SNPID rs479200 and SNPID rs480902 respectively.
6. Resequencing of the 11746 bps region of intron 1 of EGLN1 gene from chromosomal position chr1: 229598510-229610256 (Assembly-March 2006 (NCBI36/hg18).

### BRIEF DESCRIPTION OF THE DRAWING

FIG. 1. Association of rs479200 with High altitude adaptation and differences between constitution types, IE pool, natives of high altitude and HAPE

(a) Frequency of ‘TT’ genotype of rs479200 in different constitution types (K, P, V), VPK, IE, ‘natives’ of high altitude and patients of HAPE
(b) Frequency of ‘T’ allele of rs479200 in different constitution types (K, P, V), VPK, IE, ‘natives’ of high altitude and patients of HAPE. The numbers over each of the bars represent the p values of comparison of each group with HAPE.

FIG. 2. Association of rs480902 with High altitude adaptation and differences between constitution types, IE pool, natives of high altitude and HAPE

(a) Frequency of ‘CC’ genotype of rs480902 in different constitution types (K, P, V), VPK, IE, ‘natives’ of high altitude and patients of HAPE
(b) Frequency of ‘C’ allele of rs480902 in different constitution types (K, P, V), VPK, IE, ‘natives’ of high altitude and patients of HAPE. The numbers over each of the bars represent the p-values of comparison of each group with HAPE

FIG. 3. Correlation of EGLN1 genotypes of rs479200 with gene expression Box plot representing OCT values of gene expression of EGLN1 by RT PCR in ‘TT’ and ‘TC & CC’ genotypes of rs479200 in Ayurveda samples.

FIG. 4. Distribution of ‘T’ allele frequency of rs480902 in diverse IGVC and HGDP populations from different altitudes (a) Frequency in the 24 IGV populations and their altitude b) Spatial frequency map of rs480902 in IGV populations. The color gradient below the map depicts the range of observed frequency of the ‘T’ allele from minimum to maximum (c) Frequency distribution in the HGDP panel of 52 populations along with their altitudes. Diverse continental populations residing at high altitudes selectively retain the ancestral ‘T’ allele.

FIG. 5. Allele frequency distribution of rs480902 in HGDP populations

(a) Correlation of ‘T’ allele frequency of rs480902 with increasing altitude (R²=0.1056) in HGDP populations. (b) Spatial frequency map of rs480902 in the HGDP populations retrieved from HGDP selection browser. Frequencies of the ancestral ‘T’ allele that is predominantly present in populations residing at high altitude and the derived ‘C’ allele are represented by dark and light shades respectively.

### DETAIL DESCRIPTION OF THE INVENTION

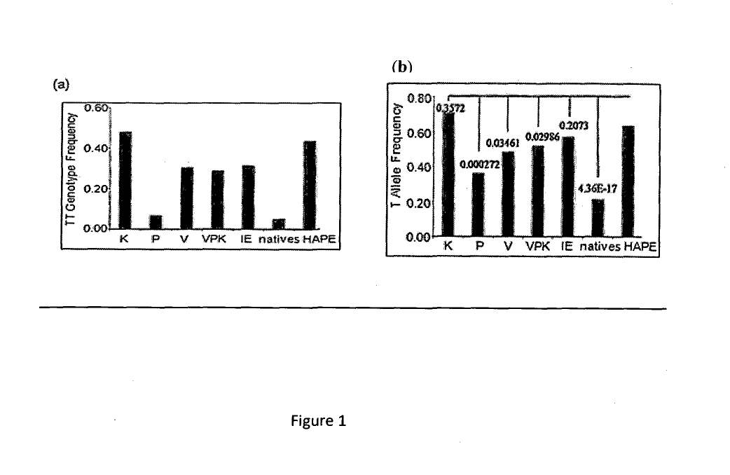

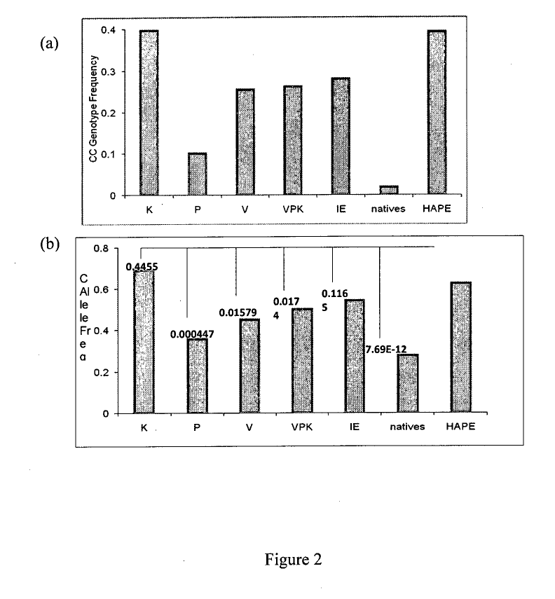

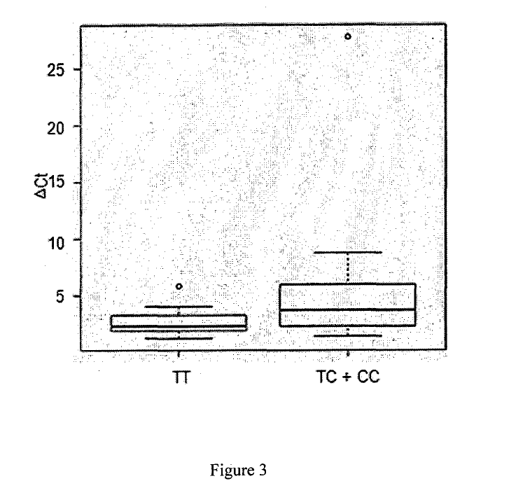

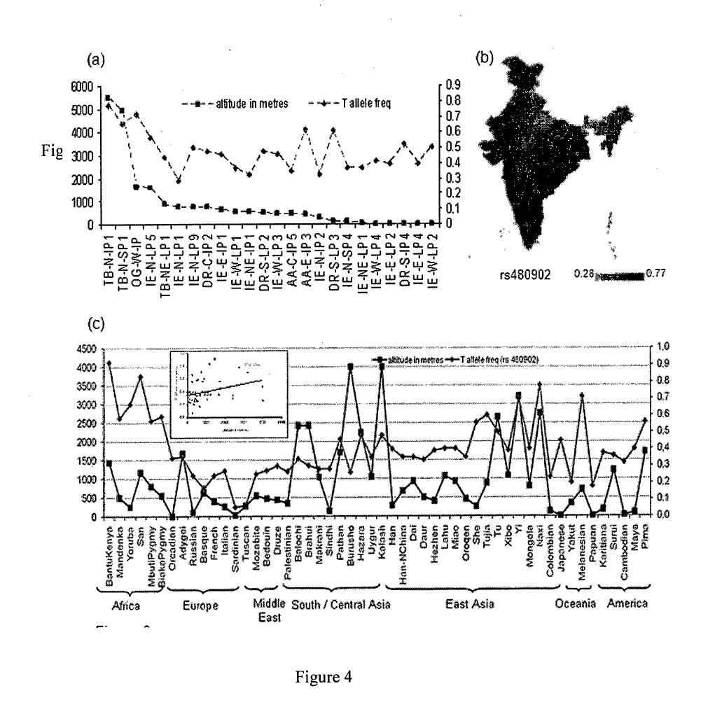

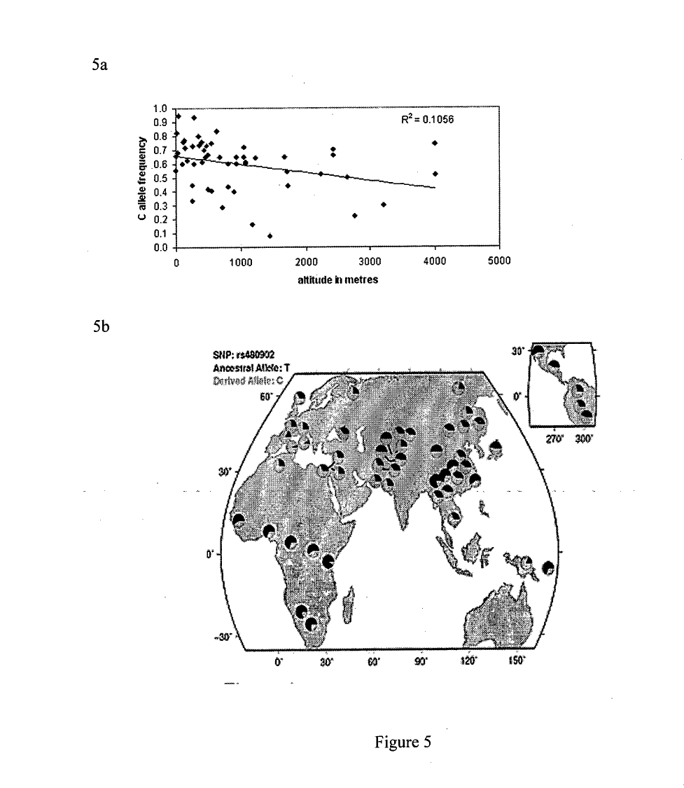

The present invention relates to detection of predisposition to HAPE and high altitude adaptation. It particularly relates with the allelic variants of EGLN1 gene (SEQ ID NO.1 and SEQ ID NO. 2), which has been related to oxygen homeostasis through regulation of HIF-1A, the hypoxia inducible factor and therefore is of importance in a large number of cellular, physiological and systemic processes. The data disclosed herein demonstrates that the ‘TT’ genotype of rs479200 of EGLN1 gene was more frequent and was found to be associated with high risk to HAPE (FIG. 1a). This ‘TT’ genotype of rs479200 was over-represented in the Kapha Prakriti types and was also found to correlate with higher expression of EGLN1 gene (FIG. 3). While the ‘TT’ frequency was significantly low in Pitta Prakriti and was found to be nearly absent in native highlander individuals. Thus Pitta Prakriti individuals harbor the protective alleles ‘C’ while Kapha Prakriti harbors risk alleles ‘T’ responsible for adaptation or maladaptation to high altitude respectively (FIG. 1b). In another SNP rs480902 in EGLN1 gene, ‘C’ allele was found to be associated with high risk to HAPE while allele ‘T’ was found to be associated with low risk (protection) from HAPE (FIG. 2b). The protective allele ‘T’ was observed to be more frequent in Pitta Prakriti individuals as well as in native highlanders while the risk allele was more frequent in Kapha types and HAPE patients. The highlight of the invention is that disparate genetic lineages residing at high altitudes share the same ancestral protective allele ‘T’ of rs480902 at global level (FIG. 4 & FIG. 5).

### Endo-Phenotyping

A questionnaire (copyright Reg No. SW-2284/2005, Reg Date 2013 May 5) for clinical phenotyping was designed on the basis of Ayurvedic literature on phenotypes and methods of Prakriti assessment (basic constitution Analysis) the details of which were provided in a paper published earlier. The phenotypic classification, broadly, takes into account parameters related to anatomical features like body build, body frame, size and symmetry of body parts, physiology, physical endurance and aptitudes.

Screening of 850 individuals for their body constitution analysis (Prakriti) was carried out. Among these 96 individuals of predominamt Prakriti comprising of Vata (39) Pitta (29) and Kapha (28) were identified and were recruited for sample collection. These individuals were of Indo-European origin, from an age group of 18-40 years (mean age ˜23±4 years) and included near equal numbers of both genders (Prasher et al 2008).

### Genotyping

### Study Subjects

1. 96 samples of individuals who were classified on the basis of their constitution types and comprised of Vata (39) Pitta (29) and Kapha (28) individuals.
2. 552 samples from 24 different Indian populations from the existing panel of IndianGenome Variation Consortium (IGVC). These populations encompass diverse ethnic and linguistic groups residing in different geographical regions and represent the entire genetic spectrum of India. These were a subset of 55 populations that had been used in the Phase I of Indian Genome Variation project to establish genetic relatedness amongst the diverse populations of India (Indian Genome Variation Consortium 2005, 2008). The populations are coded on the basis of linguistic affiliation (Indo-European, IE; Dravidian, DR; Tibeto-Burman, TB; Austro-Asiatic, AA) followed by geographical zone (North, N; South, S, East, E; West, W, Central, C; North-East NE) and ethnicity (caste LP; tribe IP; religious group, SP). Description of each population is available in the earlier study. A population (OG-W-IPI) of known African descent was included as an out-group.
3. 96 unrelated male HAPE patients from Indo-European background were recruited through SNM Hospital, Leh (alt ˜3500 m), Jammu and Kashmir, and India. HAPE was diagnosed on the basis of standard criteria, which included assessment of onset of typical symptoms at high altitude, including cough and dyspnea at rest, absence of infection, presence of pulmonary rales and cyanosis, disappearance of symptoms and signs within 3 days of the start of treatment with supplemental oxygen, and bedrest (Ahsan et al., 2004). Chest radiographic infiltrates consistent with pulmonary edema confirmed the disorder. After recovery, HAPE patients were examined to exclude any previous cardiopulmonary diseases. 96 DNA samples of unrelated Natives of Leh were also used for our study.

### Selection of Genes and SNPs

A subset of 30 genes were selected that exhibited expression differences in the earlier study. On an average 6 to 8 tag SNPs were selected per gene from dbSNP on the basis of spacing between the markers, heterozygosity, frequency and their validation status in different populations. TagSNPs from the CEPH population were identified using Tagger (www.broad.mit.edu/mpg/tagger/) with pair wise tagging at an r² cut off of 0.8. These were mostly validated SNPs with reported heterozygosity.

### SNP Genotyping and Quality Control

250 ng of genomic DNA per sample (5 μl of 50 ng/μl DNA sample) was used for genotyping using the Illumina BeadArray platform and GoldenGate Assay as per manufacturer's protocol. Raw hybridization intensity data processing, clustering and genotype calling were performed using the genotyping module in the BeadStudio package (version 2). Genotype calls were generated using the GenCall module of BeadStudio package. The genotype clusters generated for each SNP locus by GenCall were edited manually after visual inspection of clustering on two-dimensional plot. SNPs that were not found to be in HWE at 1% level of significance (P<0.01) in more than 90% of the populations were removed from the final dataset. 17 SNPs were excluded from further analysis as 5 were not in HWE and 12 were not polymorphic in the samples genotyped.

A total of 158 SNPs from 30 genes that exhibited expression differences in our earlier study and 2060 SNPS that were used for population stratification were genotyped in VPK samples as well as IGVC panel using Illumina Bead Array platform (http://igvbrowser.igib.res.in).

Genotyping of rs480902 and rs479200 on HAPE samples and natives were carried out using single base primer extension assay (SNaPSHOT™ ddNTP Primer extension kit, Applied Biosystems) on an ABI Prism 3100 Genetic Analyzer, following PCR amplification.

Genotype data on EGLN1 SNPs from 52 populations were retrieved from the Stanford HGDP SNP Genotyping Data on 650Y illumina arrays. The genotype data as well as the pie chart displaying the frequency of rs480902 across different populations (FIG. 5b) were retrieved from HGDP selection browser (http://hgdp.uchicago.edu/cgi-bin/gbrowse/HGDP/).

The altitude of each of the populations in the HGDP panel were retrieved from Google earth using the longitude and latitude coordinates provided for HGDP panel.

### Quantitative PCR Analysis

TaqMan PCR using custom designed TLDA assay (Applied Biosystems) was carried out on ABI 7900 for quantifying expression difference in EGLN1 between different Prakriti types.

### Statistical Analysis

### Population Stratification and Establishment of Genetic Homogeneity of Study Population

For analysis of population stratification variation data from 2060 SNPs was used that were unlinked to EGLN1 locus that was genotyped in the 24 Indian populations as a part of the Indian Genome Variation Consortium project and also in the 96 Ayurveda samples.

EIGENSTRAT (Price et al 2006.) was used for detection of population stratification among IGV and study population.

Genotype data for 2060 SNPs from 24 Indian populations were retrieved from the Phase II of the Indian Genome Variation Consortium data. Allele and genotype frequencies were computed by the gene-counting method.

Deviation from HWE was tested using Fisher's exact test. The distributions of allele frequencies amongst the different constitution groups were compared using Fisher's exact test implemented in R.

Fisher's exact test was also performed for estimating genotype and allelic association in RAPE. The distribution of allele was compared in affected and normal individuals using Fisher's Exact test (FET) and a p-value <0.05 was considered significant.

Correction for multiple testing was done using the FDR method.

‘Kendall's rank correlation’ was used to study the correlation of ‘altitude’ with different SNPs of EGLN1 genotyped in IGV and HGDP populations.

### Diagnostic Kits

The invention further provides diagnostic kit comprising sequences having ID Nos. 5, 6, 7, 8, 9, 10. The kits of the present invention can comprise one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least the disclosed polymorphism in EGLN1 gene. Optional additional components of the kit include, for example, restriction enzymes, reverse transcriptase or polymerase, the substrate nucleoside triphosphates, means used to label (for example, an avidin enzyme conjugate and enzyme substrate and chromogen if the label is biotin), and the appropriate buffers for reverse transcription, PCR or hybridization reactions. The kit can also comprise an instructional material for carrying out the methods of the present invention. The instructional material simply describes the embodiments of the invention disclosed herein.

### Nucleic Acid Vectors

Variant genes can be expressed in an expression vector in which a variant gene is operably linked to a native or other promoter. Usually, the promoter is eukaryotic promoter for expression in a mammalian cell. The transcription regulation sequences typically include a heterologous promoter and optionally an enhancer, which is recognized by the host. The selection of an appropriate promoter, for example trp, lac, phage promoters, glycolytic enzyme promoters and tRNA promoters depends on the host selected. Commercially available expression vectors can also be used. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide.

The following examples are given by way of illustration of the present invention and should not be construed to limit the scope of the present invention.

### EXAMPLES

### Example 1

### Endo-Phenotyping

A questionnaire (copyright Reg No. SW-2284/2005, Reg Date 2013 May 5) for clinical phenotyping was designed on the basis of Ayurvedic literature on phenotypes and methods of Prakriti assessment (basic constitution Analysis) the details of which were provided in a paper published earlier. The phenotypic classification, broadly, takes into account parameters related to anatomical features like body build, body frame, size and symmetry of body parts, physiology, physical endurance and aptitudes.

Screening of 850 individuals for their body constitution analysis (Prakriti) was carried out. Among these 96 individuals of predominamt Prakriti comprising of Vata (39) Pitta (29) and Kapha (28) were identified and were recruited for sample collection. These individuals were of Indo-European origin, from an age group of 18-40 years (mean age ˜23±4 years) and included near equal numbers of both genders (1).

### Example 2

### Subject Selection and Sample Collection

The identification of individuals of predominant Prakriti types was carried out by two Ayurveda physicians. In order to avoid any confounding observations due to population stratification the study was conducted on Indo-European speaking large populations predominantly from North India. A preliminary assessment of Prakriti was carried out on a total of 850 volunteers, nearly half by each of the two clinicians using subjective assessment and a screening questionnaire. The short-listing of individuals to be recruited for detailed phenotyping was also carried out independently. The short-listed individuals were swapped between the two clinicians and were assessed in detail for their Prakriti using the questionnaire. These comprised of nearly 120 individuals of predominant Prakriti and 200 individuals of heterogeneous Prakriti.

There was nearly 80% concordance observed in Prakriti assessment between two clinicians. Subsequently 96 unrelated ethnically matched healthy individuals with predominance of either Vata (39 individuals), Pitta (29) or Kapha (28) were identified and included equal numbers of both genders (n=48 in each case) and belonged to an age group of 18-40 years (mean age ˜23±4 years).

Peripheral blood samples of selected individuals were collected using standard procedures following ethical guidelines of Indian Council of Medical Research, India and informed consent of volunteers. Sample collection was carried out following approval of the Institutional Bioethics Committee (IBC). Three hours prior to sample collection all the volunteers were provided a similar diet with no interim intake of food, beverage or smoking. It was ensured that the subject was not ill or under any medication. Blood pressure, pulse, and menstrual cycle, if on, were also recorded. Before undertaking research using these collected samples, the samples were coded in order to maintain their anonymity.

### Example 3

### Isolation of Genomic DNA

Genomic DNA was isolated from the peripheral blood leukocytes of the selected individuals of extreme Prakriti types using a modified salting-out procedure (Miller et al 1988).

1. Acid Citrate Dextrose (ACD) Buffer
0.48 g citric acid
1.32 g sodium citrate
1.47 g glucose
Dissolve in water to a final volume of 100 ml. Autoclave and store at 4° C.

2. RBC Lysis Buffer (10×)
NH₄Cl 8.20 gm
NaHCO₃ 0.84 gm
EDTA 0.37 gm
Dissolve in 100 ml of distilled water, autoclaved and stored at 4° C.
Working dilution (1×)
For 500 ml 1×RBC lysis buffer—50 ml RBC lysis buffer (10×)+450 ml autoclaved water.

3. Nuclei Lysis Buffer (NLB)
10 mM Tris—HCL
400 mM NaCl
2 mM Na₂ EDTA (pH 8.0) (Autoclaved and stored at room temperature)
For 400 ml
1M Tris HCL (pH 8.0)—4 ml
5 M NaCl—32 ml
0.5 M EDTA (pH 8.0)—1.6 ml
Final volume made up to 400 ml (Autoclaved and stored at room temperature)

4. Proteinase K solution (20 mg/ml)
20 mg of proteinase K was dissolved in 1 ml autoclaved distilled water.
Stored at 4° C.

5. 10% SDS (Sodium Dodecyl Acetate)
For 100 ml—10 gm of SDS was dissolved in autoclaved distilled water.
Final volume made up to 100 ml (stored at room temperature).

6. 6M Saturated NaCl Solution
NaCl—35.064 gm.
Dissolved in distilled water and final volume up to 100 ml.

7. TE Buffer (200 ml)
10 mM Tris (pH 8.0)
1 mM EDTA (pH 8.0)
Autoclaved and stored at 4° C.
   DNA Isolation from Blood
Take 2-10 ml of blood and transfer it to properly labelled tubes.
Remove plasma from blood by centrifugation at 2000 rpm for 10 min.
To the remaining vol. of blood add RBC lysis buffer (1×) to make it to 50 ml.
Mix the suspension by inverting the tubes several times till it become translucent.
Keep it at room temp. for about 20 min with gentle shaking till the lysis action of buffer completed.
After this centrifuge the lysed blood to 2500 rpm for 10 min at room temp.
Discard the supernatant in hypochlorite.
Add 15 ml of RBC lysis buffer to the pellet and mix it by brief vortexing.
Centrifuge again at 1000 rpm for 10 min at room temp.
Discard the supernatant in hypochlorite.
Add 12 ml of nucleus lysis buffer to pellet and mix it by vortexing.
Add 0.8 ml of 10% SDS and 50 micro ltr. of proteinase k (20 mg/ml) and mix it well.
Incubate the tubes at 65° C. temp for 2 hrs in a water bath.
Add 4 ml of saturated Nacl (6M) sol. and shake vigorously for 15 sec.
Immediately spin the tube at 3500 rpm for 30 min. at room temp.
Take the supernatant carefully with not disturbing pellet and 2 vol. (i.e. make the volume up to 50 ml) of absolute ethanol (ie. 100%) and keep it at room temp.
Precipitate (ppt) DNA by inverting 10-20 times very slowly (or keep it overnight at −20° C.).
Transfer the ppt'ed DNA using a pipette tip to a microcentrifuge tube containing 1 ml of 70% ethanol.
Vortex briefly the tubes and centrifuge at 13000 rpm for 10 min.
Carefully remove the supernatant without disturbing the pellet.
air dry the pellet for 1-2 hrs and resuspend in 0.5-1 ml of TE buffer(Tris+EDTA) pH=8.0
Dissolve the DNA by keeping it 65° C. temp for 2 hrs and store at −20° C. temp.

### Example 4

### Primer Designing and Synthesis

### (PCR and SNaPshot Primers)

PCR primer pairs as well as snapshot primers were designed for the selected SNPs using Primer3 and Sequenom's Assay Designer software. Designed Primers were synthesized by TCGA.

| | |
|---|---|
| Primers | Sequence |

rs479200

| | | |
|---|---|---|
| SEQ ID | Forward | 5′ TATTCTGTCTTCGGCAGAGG 3′ |
| No 5 | Primer | |
| SEQ ID | Reverse | 5′ AGCAAGCAAAGAAAGGCGAG 3′ |
| No 6 | Primer | |
| SEQ ID | SNaPshot | 5′ AGGACTTTTATTATTGCTTGTTA 3′ |
| No 7 | Primer | |

rs480902

| | | |
|---|---|---|
| SEQ ID | Forward | 5′ ATTGCTTGGGAGGTTGTTGG 3′ |
| No 8 | Primer | |
| SEQ ID | Reverse | 5′ TTTCACTGGAGTTGTGGGAG 3′ |
| No 9 | Primer | |
| SEQ ID | SNaPshot | 5′ GATCTCCCAGTGACTCA 3′ |
| No 10 | Primer | |

### Example 5

### Polymerase Chain Reaction (PCR)

### Reagents:

1. 25 mM MgCl₂ (100 ml)
First make 1M MgCl₂ (50 ml)

$$\begin{matrix}W = ( 50 \times 203.31 \times 1 ) / 1000 \\ = 10.1655 gm\end{matrix}$$

Dissolve 10.1655 gm of MgCl₂ in 50 ml of Milli Q then autoclave it.
For 25 mM MgCl₂ (100 ml)
25 mM×100 ml=1×1000 mM×X ml

$$\begin{matrix}X ml = 2500 / 1000 \\ = 2.5 ml\end{matrix}$$

Then mix 2.5 ml of 1M MgCl₂ and 97.5 ml of autoclaved Milli Q.

2. 10× PCR Buffer (100 ml)
100 mM Tris pH—9.0
500 mM KCl
0.1% Gelatin

(a) 1 M of Tris (pH=9.0) (50 ml)

$$\begin{matrix}W = ( 1 \times 121.44 \times 50 ) / 1000 \\ = 6.07 gm\end{matrix}$$

Firstly, 6.07 gm of Tris is dissolved in 40 ml of autoclaved elix.
Adjust the pH at 9.0 and make up the volume up to 50 ml.

(b) 50 ml of 1M KCL

$$\begin{matrix}W = ( 1 \times 50 \times 74.55 ) / 1000 \\ = 3.72 gm\end{matrix}$$

Dissolve 3.72 gm of KCL in 50 ml of autoclaved elix.

(c) 0.1% Gelatin
For 100 ml of 10×PCR Buffer
1 M Tris=10 ml
1 M KCl=50 ml
Gelatin=100 mg (first dissolved in water)
Then make up the volume up to 100 ml and autoclave it.

3. 25 mM dNTP's (50 ml)
Stock conc. Of each dNTP'S=100 mM
Working conc.=25 mM
For 50 ml of 25 mM dNTP's
N1V1=N2V2
100 mM×V1 (ul)=2 mM×50,000 ul
V1=(2×50,000)/100
V1=1000 ul or 1 ml
So add 1 ml of dATP, 1 ml of dTTP, 1 ml of dGTP, 1 ml of dCTP and 46 ml of MQ.

4 Taq DNA Polymerase (Bangalore Genei,

5. Locus Specific Primers

For a standard 10 μl PCR reaction

| Reaction | Stock | | Final | | | |
|---|---|---|---|---|---|---|
| Components | concentration | | concentration | | Volume | |
| PCR Buffer | 10X | | 1X | | 1 | μl |
| MgCl₂ | 25 | mM | 1 | mM | 0.4 | μl |
| Forward Primer | 10 | pm | 0.4 | pm | 0.4 | μl |
| Reverse Primer | 10 | pm | 0.4 | pm | 0.4 | μl |
| Genomic DNA | 10 | ng/μl | 1 | ng/μl | 1 | μl |
| dNTP | 2 | mM | 200 | μM | 1 | μl |
| Tag DNA Polymerase | 3 | U/μl | 0.03 | U/μl | 0.1 | μl |
| Sterile water | | | | | Up to 10 | μl |

PCR reactions were carried out with an initial denaturation of 94° C. for 5 min, followed by 30 cycles of denaturation at 94° C. for 30 sec and extension at 72° C. for 45 sec. The annealing step was carried out for 30 sec and the annealing temperature was 56° C. The reaction was completed with a final extension at 72° C. for 10 min.

### Gel Check

1. 50× TAE (pH—8.5)
For 1000 ml
Tris: 242 gm
EDTA: 37.2 gm
Glacial acetic acid: 56 ml
Make up the volume up to 1000 ml.

2. Ethidium Bromide Solution
1000× stock solution (0.5 mg/ml)
50 mg Et Br.
100 ml H₂O
Protect from light

3. 10× Loading Dye
50% Glycerol
0.25% Bromophenol blue
0.25% Xylene Cyanol

The PCR product was checked on 2% agarose gel with 100 bp ladder. If a specific band was amplified then we proceed further.

### Example 6

### PEG Purification Protocol for PCR Product Clean-Up

### PEG/Sodium Acetate Solution:

PEG 8000 13.3 g

1M MgCl₂ 333 μl

3M NaOAc pH 4.8 10 ml

Final volume made up to 50 ml with MilliQ H₂O.

Two volumes of PEG/NaOAc solution were added to the PCR product and mixed properly by vortexing and incubated for 10 min at room temperature. The DNA was pelleted at 3,200 rpm for 30-60 min depending on the amplicon size. The supernatant was removed by inverting the PCR plate on tissue paper and centrifuging up to 500 rpm. Pellet was washed twice with two volumes of 70% ethanol by centrifuging at 3,200 rpm for 10 min. Ethanol was completely removed and the pellet was air-dried.

### Example 7

### SNP Genotyping Using SNaPshot Technique

We performed single base primer-extension reactions (SNaPshot™ ddNTP primer extension kit, Applied Biosystems). Briefly, the SNaPshot reaction was set using peg purified PCR product, 1 μl of genotyping primer (2 pm/μl), 0.5 μl SNaPshot ready reaction mix, 0.8 μl 5× dilution buffer (200 mM Tris, pH 9.0; 5 mM MgCl₂) and MilliQ water to make up the volume to 5 μl. The conditions for PCR were, 94° C. for 3 min, followed by 40 cycles of 96° C. for 10 sec, 50° C. for 5 sec and 60° C. for 30 sec. The unincorporated ddNTPs in the SNaPshot reaction products were digested by incubating the samples with 0.25 units of calf intestinal alkaline phosphatase (CIP) at 37° C. for 1 hr, followed by inactivation of CIP at 72° C. for 15 min. 2 μl of the SNaPshot products were mixed with 8 μl of Hi-Di formamide and loaded on 3100 Genetic Analyzer.

### Example 8

### Quantitative PCR Analysis

TAQMAN PCR using custom designed TLDA assay (Applied Biosystems) was carried out on ABI 7900. Each experiment was carried out in triplicates. RNA from all samples were reverse transcribed to cDNA using High Capacity cDNA Archive kit (Applied Biosystems, Foster City, Calif.), following the manufacturer recommended protocols. The cDNA was amplified using Taqman universal PCR mastermix (Applied Biosystems, Foster City, Calif.). It was ensured that the amount of cDNA template added to each reaction was restricted to a relatively narrow Ct range as determined by the cDNA quality control measurement of 18S rRNA.

### Example 9

### Illumina GoldenGate Assay

Illumina platform is suitable for genotyping of hundreds of SNPs in one multiplex with thousands of samples (Fan et al., 2006). This assay allows high degree of loci multiplexing, where 1536 polymorphisms can be studied together in 96 or multiples of 96 samples. The concentration of DNA samples was estimated using PicoGreen method and diluted to 50 ng/μl. An aliquot of 5 μl (250 ng) of diluted DNA per sample was used.

After selection of SNPs, all the SNPs were submitted to Illumina Assay Design Tool for scoring prior to OPA (Oligonucleotide Pool All) design. The SNP scores were supplied by Illumina and SNP score value ranges from 0 to 1.1. The SNP score reflects the ability to design a successful assay.

| | |
|---|---|
| SNP score <0.4 | Low success rate, high risk to OPA |
| SNP score 0.4-0.6 | Moderate success rate, moderate risk to OPA |
| SNP score 0.6-1.1 | High success rate, low risk to OPA |

For the present study SNP score 0.4 was selected as the lowest cutoff. The SNPs failing to achieve this score were replaced with neighboring SNP and again submitted for scoring to Illumina. The final list of SNPs with score >0.4 were then submitted to Illumina for OPA design and synthesis.

Brief introduction of the technology is presented here. Three oligonucleotides (oligos) are synthesized for each SNP: two allele specific oligos (ASOs) that distinguish the alleles of a SNP, and a locus specific oligo (LSO) just downstream of the SNP. The ASO and LSO sequences also contain target sequences for a set of universal primers (P1 and P2 for two ASOs and P3 for LSO), while each LSO also contains a particular address sequences (the illumicode) complementary to the sequences attached to beads. All the oligos querying a set of SNPs are synthesized and pooled by Illumina. To carry out the assay, this pooled oligo set (the OPA) is hybridized simultaneously to genomic DNA representing a single sample/reaction well. Following allele specific primer extension and ligation reactions, a set of fluorescently labeled universal primers (Cy3 and Cy5 labeled P1 and P2 respectively) is added and PCR is carried out, generating multiple labeled amplicons representing hundreds of different SNPs. These fluorescent products are then combined with beads on the Sentrix Array Matrix (SAM). The address sequences within the PCR amplicons hybridize to their related sequences on the beads, and the fluorescence on each bead is quantified resulting in a signal associated with a particular address sequence. Each address translates to a particular locus, and the presence of Cy3, Cy5 or both signals on a given bead type indicates AA, BB or AB genotypes.

Prior to experiment, 250 ng DNA per sample (5 μl of 50 ng/μl) was prepared in 96-well PCR plate. To ensure the reproducibility of assay, 10% of the samples were duplicated. These 96 PCR plates were processed as recommended. After hybridization, the SAM was scanned using Beadstation 500—beadarray reader. The hybridization intensities from beadarray reader were used for data processing, clustering and genotype calling using the genotyping module in the BeadStudio package v3. GenCall module of BeadStudio was used to generate genotype calls. The genotype clusters generated for each SNP locus by GenCall were edited manually after visual inspection of clusters on two-dimensional plot. All the genotype clusters were inspected and corrected manually; the threshold for GenTrain score of >0.25 was set to call a SNP successfully genotyped (Fan et al., 2006). All the duplicates used to check genotype accuracy showed >99% concordance rate. Most of samples worked well and showed high call rate across the SNPs genotyped ranging from 85-100% (average call rate 0.99±0.02).

### Example 10

### Statistical Analysis

Allele and genotype frequencies were computed by the gene-counting method. HWE was calculated using Fisher's exact test. The distributions of allele frequencies amongst the Prakriti groups were compared using Plink. Correction for multiple testing was done using the FDR method. Fisher's exact test was also performed for estimating genotype and allelic association in HAPE. The distribution of allele was compared in affected and normal individuals using Fisher's Exact test (FET) and a p-value <0.05 was considered significant.

We identified two reference genes (ASAH1 and MAN1A1) using geNORM (Vandesompele et al., 2002), a popular algorithm (VBA applet for Microsoft Excel) from a sample panel of 64 genes, as internal control to measure relative expression of EGLN1 between the constitution types. Briefly, this method identifies the most stable reference genes from a candidate panel of genes. geNOrm calculates the gene expression stability measure M for a reference gene as the average pairwise variation V for that gene with all other tested reference genes. Stepwise exclusion of the gene with the highest M value allows ranking of the candidate reference genes according to their expression stability. Gene expression normalization factor for each sample based on the geometric mean of a user defined number of housekeeping genes is then performed. Low or High mean ΔCt values were inferred as up-regulation or down-regulation respectively. Difference in expression of EGLN1 with respect to rs479200 genotypes were compared using one tailed t-test. We used ‘Kendall's rank correlation’ to study the correlation of ‘altitude’ with Ireq of the T′ of rs480902 in both IGV as well as SNPs of EGLN1 genotyped in HGDP populations.

### Advantages

The present invention is useful to identify individuals who might be at risk of developing HAPE when they travel or are deputed to high altitude places. The information may be useful for molecular diagnosis both at genetic and expression level, prediction of an individual's adaptability to high altitude, endurance, susceptibility to HAPE and other diseases where hypoxia is implicated for e.g cardiovascular disorder, cancer progression, asthma, neurodegeneration, ischemia, stroke, liver diseases; skin diseases, arthritis etc and also responsiveness to PHD2 inhibitors.

We claim:

1. A biomarker useful for predicting predisposition of an individual to high altitude adaptation and high-altitude pulmonary edema (HAPE) characterized in having single nucleotide polymorphism C/T at position 27 in SEQ ID NO. 1 and T/C at position 27 in SEQ ID NO. 2 of the EGLN1 gene.

2. The biomarker as claimed in claim 1, wherein the frequency of occurrence of “C” allele of rs480902 having SEQ ID NO. 2 in people who develop high-altitude pulmonary edema is significantly (p value <0.05) higher than that of people residing at high altitude.

3. The biomarker as claimed in claim 1, wherein the frequency of occurrence of C allele of rs479200 having SEQ ID NO. 1 in people residing at high-altitude is significantly (p value <0.05) higher than that of people residing at low-altitude.

4. The biomarker as claimed in claim 1, wherein frequency of occurrence of “CC” genotype of rs480902 having SEQ ID NO. 2 in people who develop high-altitude pulmonary edema is significantly (p value <0.05) higher than that of people residing at high altitude.

5. The biomarker as claimed in claim 1, wherein the frequency of occurrence of “T” allele of rs479200 having SEQ ID NO. 1 in people who develop high-altitude pulmonary edema is significantly (p value <0.05) higher than that of people residing at high altitude.

6. The biomarker as claimed in claim 1, wherein the frequency of occurrence of “TT” genotype of rs479200 having SEQ ID NO.1 in people who develop high-altitude pulmonary edema is significantly (p value <0.05) higher than that of people residing at high altitude.

7. The biomarker as claimed in claim 1, wherein the frequency of occurrence of T allele of rs480902 having SEQ ID NO. 2 in people residing at high-altitude is significantly (p value <0.05) higher than that of people residing at low-altitude.

8. Primer useful for amplifying biomarker as claimed in claim 1-7 having sequences selected from the group comprising of:
SEQ ID NO. 5 represented by 5′ TATTCTGTCTTCGGCAGAGG 3′ which is a forward primer;
SEQ ID NO. 6 represented by 5′ AGCAAGCAAAGAAAGGCGAG 3′ which is a reverse primer;
SEQ ID NO. 7 represented by 5′ AGGACTTTTATTATTGCTTGTTA 3′ which is a SNaPshot Primer;
SEQ ID NO. 8 represented by 5′ ATTGCTTGGGAGGTTGTTGG 3′ which is a forward primer;
SEQ ID NO. 9 represented by 5′ TTTCACTGGAGTTGTGGGAG 3′ which is a reverse primer;
SEQ ID NO. 10 represented by'5′ GATCTCCCAGTGACTCA 3′ which is a SNaPshot Primer.

9. A method of preparing biomarkers as claimed in claim 1, wherein the said method comprises:—
a) isolating genomic DNA from human subject;
b) designing and synthesizing forward and reverse oligonucleotide primers having SEQ ID NOs: 8 and 9 for positive strand of intron 1 of the EGLN1 gene;
c) amplifying positive strand of intron 1 of the EGLN1 gene having SEQ ID NO. 3 using primers synthesized in step b to obtain biomarker of sequence ID No. 2 having SNPID rs480902;
d) designing and synthesizing forward and reverse oligonucleotide primers having SEQ ID NOs: 5 and 6 for negative strand of intron 1 of the EGLN1 gene;
e) amplifying negative strand of intron 1 of the EGLN1 gene having SEQ ID NO. 4 using primers synthesized in step d to obtain biomarker of sequence ID No. 1 having SNPID rs479200;
f) determining SNPs in HAPE patients and native highlander subjects using snap shot primers of SEQ ID NOs: 7 and 10 for SNPID rs479200 and SNPID rs480902 respectively;

10. A method for detecting high altitude adaptation and predisposition of an individual to high altitude pulmonary edema, wherein the said method comprising the steps of:
a) isolating genomic DNA from human subject;
b) designing and synthesizing forward and reverse oligonucleotide primers having SEQ ID NOs: 8 and 9 for positive strand of intron 1 of the EGLN1 gene;
c) amplifying positive strand of intron 1 of the EGLN1 gene having SEQ ID NO. 3 using primers synthesized in step b to obtain biomarker of sequence ID No. 2 having SNPID rs480902;
d) designing and synthesizing forward and reverse oligonucleotide primers having SEQ ID NOs: 5 and 6 for negative strand of intron 1 of the EGLN1 gene;
e) amplifying negative strand of intron 1 of the EGLN1 gene having SEQ ID NO. 4 using primers synthesized in step d to obtain biomarker of sequence ID No. 1 having SNPID rs479200;
f) determining SNPs in HAPE patients and native highlander subjects using snap shot primers of SEQ ID NOs: 7 and 10 for SNPID rs479200 and SNPID rs480902 respectively,
g) computing the frequencies of TT,TC and CC genotypes in the populations of step (f) for establishing the association of the genotypes with high altitude adaptation and high altitude pulmonary edema;
h) predicting and statistically analyzing differences in the distribution of the allelic variants (T, C) in the population wherein TT genotype of rs479200 and CC genotype of rs480902 in EGLN1 gene are at high risk to high altitude pulmonary edema and CC genotype of rs479200 and TT genotype of rs480902 in EGLN1 gene are at low risk to high altitude pulmonary edema.

11. A kit for detecting high altitude adaptation and predisposition of an individual to high altitude pulmonary edema in human subject, wherein the said kit comprising:
i. primers having SEQ ID NOs. 5, 6, 7, 8, 9, 10;
ii. suitable reagents;
iii. instruction manual.

12. Use of Biomarkers for predicting predisposition of an individual to high altitude adaptation and high-altitude pulmonary edema (HAPE) characterized in having single nucleotide polymorphism C/T at position 27 in SEQ ID NO. 1 and T/C at position 27 in SEQ ID NO. 2 of the EGLN1 gene.


SEQUENCE LISTING (10 sequences)

SEQ ID NO: 1 - DNA, 52 bp, Homo sapiens
  misc_feature (1)..(52): C/T AT 27
cagaggacttttattattgcttgttatgtacagtattagtcatatcttgcta

SEQ ID NO: 2 - DNA, 52 bp, Homo sapiens
  misc_feature (1)..(52): T/C AT 27
tctccaagtgatctcccagtgactcacagacacattaatgtagaccagaagt

SEQ ID NO: 3 - DNA, 46898 bp, Homo sapiens
  positive strain of Intron (1)..(46898): snp rs 480902 at 21782
ctgtaataatgataataatgattattaaagtccatgtataaataaaaagagagaacagctaataaatcagatctctgatttttggctactgatttacacttagattcttctaataaaaataatattttgtagataagataaggctattctacattaacatttatagggaacacaagataggttgatgaacaaggactggtgaattctgcttcacagtgataactccaaagatcagaaaaatttcttggtcctactttgacaatctttataaatacagataatcaagatcaagtacgtttttgtcctgctccatgggagatttcaatcttccttgagctcacctcagaacacctactttaccatctgataggtgaaccaacccagtcaacaccccatctgacactgtctctgaatggtaccaattttttttttttttgagacagggtctggctctgttgctcaggttcgagtgcagtgatgcaatcatggctcactgcggcctcaacctcctgggctcaggtgattctcccacctcagcctcctaagtagctaggactataagcacgtgtcaccaggctggttaatttttgtattttttgtagatatggtgtcttactatgttgtaatagggataatttaggaggtcaactaaactccaaacattaactcatttagtgtcagtgatttttttttaaaaaaaggactaaacaaattagcttatgtttaccctctttcaatcagaaagaaaattatttgctcagattataacaagatatggttcagaagactgttggatgttaagcttaaaggtagaaaagaggcccaaaataaaaagcacaaaataacttccatattctaggtgtacatgataattaactgaaattggtaatggtattttttaaaaaattctcaggaattatgaagaaaacaaacaaagacgttcaaggttatgagctattcttgtttttgtttgtttttttacagagtcttgctctgttgaccaggctagagtgcagtggcacgatctcagctcactgcctccaccttccaggttcaagcgattctcctgcctcggcctcccgagtagctgggattacaggtgcatgctgccaggcccggctaatttttgtatttttagtagatacagggtttcaccatgttggccaagctggtcttaaactcctgacctcaagtgatcctcctgcctcagcctcccaaagtgctggaattacaggcttgagccaccacgccacccggtttgttatgagctattcttgaagtcctttatttcttcatataataaagtcaatgaattagtataaagttatcaaacagtaaccgaattgttctactatcatattaaaagaaaaatgctaatttttaaaactcacttcccatccctaagaaactaagaggcatcttaaacctaaatccttatttaaaaatcctgtttccctagacactctgaattcactgctctttataagcagcagaatttttggagaacattatatatgtctctgtttttcctgattccagacagacttaacaagttccttctttcagcactggaggttggtaacaaccaggagtgtggcatgggcttagtgggtttaagtgctcaataaatgatgagcccaaatgattttcttgtcactagctacccaggaaagaagtttaccttgaatagtaactgtaagagggtactaatttaacaatgcaattactccatttgtggttaactgaagtaattatgatgaattacacaatatattgttattcatgggaaagaagggggaaacctgaactctacatctcaaaaaagttaatttgggcctttccaactatctggtaacttcagcatctcttgtgtaaagaaggaaggggaaaatatttgtcagtcactcaccccatcacatcccatatatccatttaacaaatacctcttgaacatcgactatgtatatgaaaggcactggggctaaaacgatgaaccaaagagaaaaggtcccttgcactcatttacatagtccctccttataagaagttttgctttctgtgatttcagttacctttggtcaacttagtccaaaaaatttaatagaaaattccagaaataaacagtttataaattgcacaccattctgagtagtgcactgaaataccttgcagtcctgccccgttttgcctgggatgaatcatccctttgtccagcatatccacactgtagatgctatccacccattagtcatttagtagccttctaggtcatcagagcaatcgtcgtggtacttgtgttcaaggaaaccttattttatttaataacagctccaaagtgcaagagtagcgatgctggcatactgttataattgtcctattttactgttgttgttaatctcttactttataaattaagctttattataggtatgtatgtacaggaaaaaatatagtatattaggatttggtactatccacagtttcaggcatccacttggtctcggaaggtattgccctcagataagaggggactattgtactatgaaatctggtctaacagcataattagccaataaacagatatataatgtaatgttaggtagtgaaaagttgtattttcttaagatatgaaaaaaattaggtatatagagaaagatgacatgatattcttgaacagtggttgacatttgggtttagacttaaatgaagggagcggcaagttgggcaaatctctgaagagagagcaatgtggcagagggaaaatgagtgctcaaaggctgaacaaggcaggaaagagcagcaggccagtgtggctggaacacagcgagcacgggccaatgtgatcagagaggaaccaggtaacctagagctgcagggcttggtaagccacaggaaggcctttggatattgtgctaagaagggccaggaagccaacagagaatgtatctgactaatgttttgagaatatcagtttcaagtctgtatgaataaaaacaaccacaacaacctcaatatctcttaccacctaacatttatgggacatttactgtttgccatgtcctatctaagtaatttgtgcataacaagttgtctgacacaaccatgtgaggtaaacagtatcactgttctcatcctcattcctctaaactgaggtttagaaaggctaattagtattcgcaaagtcacatgacagggaatctggttccagaacccatggaattaaccactgtactagactgcaggaagcaaagagtgaaaagagaagactaagttagctattaaagtttccgggcaagaaaccaaagaggctgagcccagagtagcaaagtggggtgtattctgaaggcagaaaaccaacatgatttgctaatgagtggagagtaagaagagtcaagaatgactacggggttttcagcctaggtaagtaaatgaatggagtcactgtacatgaaaatggaaacactaggtgaaagagcagttttaattacaagcatcgggtaggagaggagtttatcgataactgtcaaaatgagccagacatcccagaatagaacaaaactgagggcaccaggattatcccccaaccggggatctgtttgtattattcacttctaaactctttccttagagccctggagccctagggaggtatctaggggtaggggactaattgggaatgaagtgatatgagaagaggccaaacaagcaggttttaaccccaaccttaaactagaggcagtgcttagctgtttcctatacagtatttgaattccaagtgaggattctttgaaaaaataaaaggcagagtaaagaaaagttttaaatctcacgttttgaagaaaagttttaataaatttaatgtaattgtgttaagttctataattcccagtatagctcaattatcaccatctaaatgtattatttaaaagtggccattaccactttagtcttatctggcattccctaggtcccttgtgctctgctctggtgacaggaatactgaaaaatgaggagaaaggaagaagggagaggggatgagtgctccacagggcctgcatggatctgccaacccaattaacttgccaataccagccccaggaaaggcagcagttcatgattataattgtaaaaagaataacacgaaaaataccaacataaccctataaaccagaaagcctgaaaaagaaacaatgcaatccaccactatataattctgaggtaaattcctaatttgccaccttatagagacaccaactttaaatgtttcaatttcccatcgagaaaaacctatttatacataaacaaaacaaaacaatactatttttttcttttctgatatgaaaatatatatgattaaggatgttcttttttacgttttcagagacaaggtctcacgacgttgcccacgctggtcttcccctcctgggctcaagcaatctgcctgcctcggcctcccaaagtgctaggactacaggcgtgagacaccgtgcctggctgattgagaatgttcttaagataggaaacagcataaatttgtttttcactctgagaataaaaggaaggaagaggtccgcaatgcctctgaggaatctagagacatttatttctagcattagaaggcttcaatatgaagctgaaccaggaaacttatttttcttttttgagctgaattattttaaaacagggatacaaagcttagagaactaaactttgatgggttacagaactattatttttaccctgaatgaagggaaaaggtttactatctgaaaatttatcctattaaactatattatcaaagcactatgctttgacatagcactatgccttttgtagaggctgacagctaacatttactaatcacttataggccaggcatggattacctcggtcattctttacagcaaaccttaggtaggcacaatttctccccccattttagagaccagaaaactaaactggagtgaaaagaaaaaaattgccgaaggtcagagactctaaatggcagaggcaggatatgaactgaggcagagaaaaacaacttccttctgtattcttcttcaattccttgctccagtaaggcatgtatatagtccacccaagagttcagatgaaattggtaatccagactcgtaagcggcccaccacctgctgcagtggcacatgctcatgcctaatgcccaattctcccatgtcaagatgtacagtaagtcctcactaatagcctcgataggttcttggaaactttatgtgaaacaatgtataatgaaaccaacttaagtttctacagcatatttgtggtcataaaacattgccaaacttctaaataaagaccaaaacacttctaatggtaaaccttgaaataaatgtgagctatatacacatttaagaaagattaataaaaacaaggtaattatttatgccattagtccagttttagggttgcaggtggctggggcctatcctgggagctcaaagcacgagatgggagccagccttgggcaggacgccattctactgcaaggtgcactcacacacacccatattcactcacactaagataacagatacgtcagtccacatcccaaagatgcacacattgggatgtgggaggaaactggagcacctgaagaaaacttcgcagacatggggagaacttgcatgctccacacagacattggcctcggccaggaattgattctttatccctcatcaacattataacaaaacgacactgaacagaataatgttattcaagaacctgctgtactgcacatggtatttttcagttctacagttaaatctttttaagtctcttccctgcccacttccaaagcacacatacacatcactatcactaccttgggggtctacaaaaatctagttcaaatacggaagaaaacaggtcatgctggaaggccatccaggtggggatgttcaaagggcagttggaaacttgtgtggaactccaaaaaaaagtcatgactggaaatgtaaatttaggagccatcttcgcatgggatagctgaagccgtgtgcctgccacagattgccaagggagtacagagagaagagaacagagaacagattgtcatgaaataaagtcatttagaataagaactccttgaatggcgtgaacccgggaggcggagcttgcagtgagcggagatcgcgccactgcactcaagcctgggcgacagagcgagactccgtctcaaaaaaaaaaaaaaaaaaaagaataagaactccttaagccttgctgctttcttgatcacagtaaacaactcagtggtcaaaagctgacttattataaatgatttctgactaattttttgactaattcatttctaagtgtaaatgataggtatatatatattttaaaaagacatataagggcatatagtttccttaaaaattagaaaaatgctattataataatattgtgatcaacaatagttttggagcttaatctttgtaaatccttgatattttcctaaaattaaattactataagtgaaattattaggtcttaaagctttctgattgctgaaatgttctccagaattattgcatgagcttatattcctactggctttgtatgagtgcacacatctttttaaccatttgccaacccagcatagtaagttttaatctttgaaaacagctcaatttgataggtaacatttatcttactaatgttttaattacatcttatatttttaagtcagcatttttcatattttattggccatttaattcttcaactgcttgctcatatcctttatccacttttctactgggttaatcttttccctactgagttattattattattttagagacagggtctcactctgtcacccaggctggagtgcagtggcgtgatcatggttcactgcaactttgaacatctgggatcaagcaatcctcccaagtagctgggagtacaggtacacgctaccacacctggctaattttttaatgttttctagagatgggctctcactacgttgttcaggctggtcttgaacttctggtctcaagtgatcctcccactctggcctcacaaagtactgggattacaggtgtgagccaccacacctggccccctactgatttttaagagctctttggttgttaaccgctgtcatatgacacaacaacaatttcaacctttttactctgcttgtgtggaagagtatgttaatacaaagtttttattatcggtacaggaaggcagaatataattatattaattatcaatgtaaagtttttaaaaatccaagattcttcccatgttcatagaaatatgaaaaatacagaaaaacaatgaaagattattgaagaagaaatagcattgatccaaatcactctccaagtggcccaagaattatgaattgtttaataatctaacaccaaactactttccttattaggctctaattctagcccatgattaaaaaaacttaaatatacacttaaaaatcaacttaagtatgatgctcccgacagtataatctagagatatgtcatgatgccttagtactctctgagctttcatttcccttttagtgaagaattgataaagttatgctttacaaataagaaattcatctggtgaaacacattatattttgagctgtctagctctgctgtcagaacagagatctacaatttgttaacctcagctataatcttaccagcactgcacaactctttcttagattcccaagcagggctctgtatttatatcacacacaccactcccactgcaaacttcatgaggaagacagagcaacctctctccacaaggagactggggatcctgagcaggtctcagcttgatccccacccagggaattatacaacctcattctagcccatgcaaggaagtatcactgttaagacagatattctgacagctatggcaaacttgctatattattaatataattcgttggaagagtctgcaattcggcattaaaaacaaaaatacaaacagaacaaaacacttagctgcttgacattacaaacatacattctggcacttcttgaggctcactgagcaggtactgcactaggcatgaggatacagagattaaaagtaaaacatctgtttcaattccagggtctagaagagttacgatttaaattccttaagttattttggcacaaacagtgtaattattatttgttttaatggactagaggacatgagtgagaggttcctgacattcatcctggagcacttagttaacatgcaagcccaagactggatataaataatcatcagtaaatgaggaaagcttgtaatcaaaaaccctactccagtgagacaactcaagaagccttatctcatctcaaagccagagaatttagtaattaacatagtggatattagaaattatgacagtattagagaagaatggtactcaagaaaggtgttatggctacacactgtatagggaccactgctcttacatgaacatatacatgaatctagttaatctaatggcctactattagggactgtaccctgagccttatgactcctgctggttctgttagttccccataaaggaattctgttatttgattccaattaggttcagctgaatcctaaaagcacactagcatttttgccttaaataaaccttccataaaaggtgcttggcaggccgggcatggtggctcaagccggtaatcccagcaccttgggaggccgaggcaggtggatcatctgaggtcagaagtttgagaccagcctggccaacatggtgaaaccccatctttactaaaaatacaaaaatcacccaggtggggtggcacacgcctgtaatcccagctactcgggaggctgaggcaggagaatcgcttgaacctgggaggtggagggtgcagtgagccgacatcgcaccactgcattccagcctgggcaacagagcgaaactctgtctcaaaaaaaaaaaaaaaaaaaaaaaaaaaaaggtgcttgtcagatgttctaaaagcatagagagtaggaattttatccacctaataaaacagtatttttgtgcttgcttcctaataaattttttttgtgtgtggcagaactcagtgtgagttttgctttcacagtaacatgccatttagaagctcggttctgggggaagagatttgtaacactcatttaactgtagcctccaaagaacagcagtacctctcaataaaaacctaatacccgtaaacaactactggctcaggtcaggtcacaacaggcaaacagttatgcaaaggcacacagtacagtgtaataaacaccagaacagagtcttacttacacaaagtacccagacagcaaagacataattaactttgtcacgggaatggggatcagaaaagactttatagagaaagtgacaaacgtgcagaattttgaaagacaaaaatagaagttttccagagtagaagattctgttctgtacagggaaaagaatatttgcaaaaacagaatcaatatggtgccttgagagaaatgcaaatagaaaaactgttggaagatgaagtgcagtgtggagcaatagggatggggcaggacagacaggcaggggcagctcaaaaaggctctcactgactcactggggaactttaccttacaggccagtgttggccaactgtgttcagtaagaatcgcttgggttacttcctaaattatgatgattcttatgccctctgcttgaaattatgacttatcaggtatggcatgaggcctgagatcccgtacttttttttcttgacaattattccttcataagagatcctgtactttaaacaagcacttcattgtaattggcaaaaaatcaaaaataaagagttttgaataataaaatcatttttccattttacaaacatcactcaatctacttgtagaggatggcccagatgccacgaaataaaaggttatggacacactgctaatagatgagagtcagaataaaggggagtgagaatggaaaggacagggcagcttagagcaacagtaagagtgtgaaacagaaaggatatggtgctggcgagagagaaagaactcatttaaaaaaaattaaaccctaaaaatatacactatgtgttcattcaacaaatattttctgagcacctactatacgtcaggcactgttctagatcttggggatacactacttaatagaaaagatacaaagttactgccttcagggaacatttattctagcaggaaaagaaagagaaaaaattaagtcaatatatagcataatataagaaatggcagaagacaagcattacgaagaaaaaaaaaaacacgcaatagggaatataatgctagaatgtaattatttatagggtggttggtcaggtaggcctgactgagaagttgacatttaagaagaaatgaatgttccaggcagaaggaataaatggtgcaaagaatcaggtggcaatgggcctggcatatctgaggaaaaagagggtcagtgtgactggagaggagggaggaaagcagacaacagcgtaagatgaagtcagacaggtaaggggttggggacgcacatcatggagggttttagagaacactataaagattaagtcaccggaaggcactgggtagtctggttgccatgtagagcaggggctggcagaccacagcccactgcctgtttttataagtaaagctgtattggaatgcaaccacattcattcaattacatattctctatggctgcttttgtgccccaactccggtctagcagctatgacagagaccgtgcaggccacaaaacctaaattttttaccatctggcccttgacataaaaagtctgccaagtcctgatgtgcaaaatagactgaaggctggcaagagtggaaggcagaagagtagttagaaggctaccgcataaatcaggtgtggtacaacgatcacttggaccacagtggtagcaatggaggtggtaagaagtgctgggatactcatactatcttaaaggcagaataaagcaagatgtccagaaagactggtatgagatataaaagaagatgactctaaggttttggtttaagcaatagggaaaatgtaattaccataaacagatgaagacagcatatggtgcagcctgagtctggacaatgggagagatcaggagttcagttttgaacatgttcagttagggtatgtgtaaggcatagtgctaggaagtgtgagtagaacagagaggaaaactgatcattccaacacagatgacaattaaaatgaccagtccatgcgctgactacttcactggggagaaggttgggatcaggaaggactatctgaaaaatgagttttgaaagagtatgaattagccaggtaaggaaggagagggggcatattccaggaagagaaaaggtcatggtgtgtgtgtagacacatgctgtgtaaactgcaaacaagggtgaacctgacgtgtgagtcaaggaatgtgacaggtaaggctggacatagctctggccccttcccaaaagcttagtaagccccaatgatgaacttgggttttagtgggtaacagggagccactgaagaggtttaaacaaagaagtgacacattcagactagaggttttcattcacttcggtaggtgactgagtggcaccatttttgtagtacagctataaagcagtaagttgctaagactggccagaaaacatgtaaatcaggctcttaagaattcagtaactgcaataaaaaggagctgatttttttctttttctatttttgttttcctgagacacggtcttgctctgttgctggagtgcagtggcgtgatcatggctcaatgcagcctcagcctcctgggctcctgcctcagcctcctgagtagactagctaatttttaaattttttatagagacagggtcttacttactatgttgcccagctggcctcggacttctggactcaagtgatctgcctgccttggcctcccaaagtgctggaattacaggcacgaaccaccatgcccggctggttttttttttcaataatagcgtaactccctttgtaggcagataaccaaacacttctgaaaagtttttttgtggcatgatggacaaagattcaagtcacaactttcatcagagtacaagttctatttgtatagaactctagttcacagcatttacaaataattatctcatttcaattctgcaacaactccatgaggtattcagagaaatgactatctccattttacaaataattttatctagtcttgttttaagagctgaagtgtttaatctttgcatccataccactgcgggagagtgtggttaatgtaaggacaagatccagtcttagaaaataatgatgagtctgtttcatctaacattaacctcattctacttttatcactttgaattagtttaatgttatttgttttttcacatcattcttagaaaagttacttaattatttatatattgccacacctaaacacactcatttttttttctgggtttccccaaccagactgtagactccaacagaggatgaaccagctcttatttattcttgtactcctagtgccaaacacaggcactcaaatatctactgaataaatcaattaatgatttaccttgagagaacagagatgatagattagacagataaagaagagtaacataaacatctttaatacaattttaaaacagaaggtgggttttaccatccattgctaaagattgatagaagtactgctagcttgtaaaaatatatatacatatattttagacacaaggtctcattctgtcacttaggctagggcgcagtggcttggatcatagctcactacaacctcaaactcctgagtgggagtaatcctcctgcctcagcctcctgaacagctctacaggtgtgcaccatcacacccagctaattttttaaaatgttttgtacagatgaggtctcgctatgttgcccagggtggtctcaaactcctggcctcacgcaatcctcctgcctcagcctcccaaagcactgagactacaggtgtgagcccctgtggctagtcaaaaaaaaaaaaattgaagtttgacaacttgccatttgcatgagatgtcaaaataacaccggcatgctatatatgtatacacacacacacacacacacccccctaattaaataaagacagaatactaaattatgcctacagatggttttcattcagtaccatgtgtttttattttggccctagatgcaacctatttttttaatttttttttttttttttgtgacggcgtctcgctctgtcgcccaggctggagtgcagtggcttgatctcggctcactgcaagctccacctcccgggttcatgccattctcctgcctcagcctcccgagtagctgggactacaggtgcctgccaccacgtccggctaattttttgtatttttagtagagacggggtttcgccgtgttagccaggatggtctcgatctcctgacctcatgatctgcccgcctcggcctcccaaagtgctgggattataggcgtgagccaccacacccggccgcaacttatttttaaggatgtttaagtatacctagtgagtggtttagaaaaacagactgagtacaagattttcataacaactattaagaagtcaagttctctatatccttacatcccatcaatatctgtcaggcatactgtcagtaactaccaatatccaatctctctctcaaatgcttgttcccaccacccaccagaacatccatctataccactcttctgcctctcacctattcttcagtcacctgtttctttgatccacagttttctggtggcccaggcttaatctttatgtcacaatgtttgctgaacctgttaagtgatctaccctgctcatctgatcaatatcattgtatgtaaatctactgtaggcagatctttacagattgctatacactattacatctaaatgtcttgttgacaaaagaggaagtttattttggttgtagagggtgttagaacccagtgactctaagcaaagtgattgatgggccataaaacaaaaattgagtaccactgctaaaggatatgactaatttaaagagaatgttgcaagaagtctaaggacagaggatggaggcctcacatttgtaattaaattgagagaactgtaagaaaattaagagggaagaaccaactagatacattttttaaaatcttgaaaaatattctcacatatgtggttgataagacggtgaaatgtgagctgggcaaactaacattaatagttagtttctctatgaatcctacatccaggcctctctgccagcctgccaggaggttctactatctctagcctgtagctagtgtctgtctctgtctcgctctctgtctgtctcagtctcactctgtcgcccaggctggagggcagtggcgtgatcttggctactgcaacctctacctccctggttcaaacaattcccctgccgcagcctcccaagtagctgggattacaggcatgcaccaccatgcctacctaattttttggttttttgttgttgttgttgttgttttgagacggcgtctctctctgtcgcccaggctgaagtgcaatggcgtgatctcagctcacttcaacctccgcctcctgagttcaagcgattctcctgcctcagcctcccaagcagctgggactacaggcgcccaccaccacgcccagctaatttttatatttttaatagagacaggatttcaccatgttggccagactggtctcaaactcccaacctcaggcgatctgcccacctcagcctcccaaaatgctaggattacaggcgtgagccaccgcgcccagcctagctagtctatatttttatcaatgattttgagtcacacaaataaaggaatcttattaatgtaacctttctcaataggaaaatatacagctcatacaaagctgggaaaagtagccaatatcacaaataaaagaaagaagattcaatgtgatctcaattgaatcaacatgatgaattctaataaagttaaaaataaatcctactttcaaaatggcaaaaatcaaccatactatacagaagtaataaccagcagcatttactgacaaattactatgtgttaataactttacatttattctttcacttaatctttacaatccctggaaggcaggttttattctcatcttgcagagtaaactataaaatgagggtaagacagcttggcagaatagagatctttgaagcttgtatccatttctggggacacattagagggcgaacgttgacaaaatgaaaaaaaatttttttttaaagaaaagtggccaaaatagtgacagcaatatccagcgagtaacagtggcaataaatgagtaggtttagcctggagaagacacattcctcatggactgtaatctctggagtacagggaccaagtcagataaatataccacatcttatgtagcaccttctagtttgtaaaactgcttttcaccttcacaaaagagagcatgaagtaaaaggaagaacacaaagattaagaacagtggggctctgaccgggtgcggtggctcacgcctgtaatcccagcactttggggggccgaggcaggcgggtcacgaggtcaagagattgagaccatcctgcccaacatggtgaaaccccatctctactacaaatacaaaaaatcagctgggtgtggtggcaagcgcctgtagccccagctacttgggaggctgaggcaggaggatcacttgaacccgggaggcagaggttgcagtgaactgagatagcgccactgtactccagcctggcaatacagcaagatgctctctgaaaacaaacaaacaaacaaacaacaacaacaacaaaagaacagtgggactctgaagccagatttcctgcacatgaagcacacgactctgccacttactgactgtgttactttgggcaaattattcaacttctctgtacctaactacccttgtctgttaagtgaaaataacaatagtataccacatgcaaagctaataaactagtacataaaaatgcttaaaacagtgtctggtgcactttttaaaaataatgtgtatggtaggtatgactaatggctgtggaccttggggaagtcccaagccttcagcgctgtttcctcacttgaaaaattagggtatcactatctacctcacaggggtatgaatcaaataagacaatacaggaaaatgttccataagtcttaattccttccttctttcataaactctaagagagtctactatgtgccacgtactatgctaagtttaatctttatagtctcaatcctcaccacaactgtgtaaatgtctactactattattctcattttgtagacaaagaaatgcggcttagaatggctaacttaactgaggtcatgctcctaagtggtcagatagactgtgaacccaaggttgttggactctatattccagttttgggcctccatatatgcacattccctcatgtacacatctttgcagctaaccacatgcctagtaaaatgcctgatagattgaagcgattcaataaatgttcctttaatcaaataaatgaatgttgtctgtcttcaagttgctaataccgttttgaggaaagaagagcataaggcagcacaggcagaaatgcctgtccttgctccactacttctaaatgatggtgagatcttagacgctatcatttgggcctggattatctttactattattaattttttttttttttaagagacaaggtctctgtcccccaggctggagatcagtggcgtgatcatagctcaccgcaacgtcaaactcctgagctcaagcaatactcctgccccagtctcctgagtagctgggactacagacgcaagccaccatgcccggctagtgtgcgtgtagacaggaactgattcaccacgaccgacgcgtgtgtgtgtgagacagggaactcattctgttttttttttgtgtgtgtgtgtgtgtgtgtgtgtgtgtgtgtgtgtgtgtgagacagggaactcattctgttgcccaggctggtctcaaactcatgggctcaagtgatcctcccatcttggcctccccaagtcctgggattacaggtgtgagccaccatgcccagctggtatatctttaaaataaggaacctggactatgctatcttccaatctagctctaagactatacatccataaggcagtgcttctgatctagcagagatttaaaataaaaacactagaattttaaaagtggaaaagatcataaaggtctggcccaaactctttagagaggggaatcccaaaggacaaaaaggggtctcctgatttccaatcctgcactgcttcctttatgacgacacagatcagacatttcagacaggattaaaaaaatataattggcaccgcagagccccaaatcatgaaaaccaaaataaaatggtactcggtaacttttattagtctaaaaactcaaactctgttacaaaacatatggttttaatgcttaaaatacgcatcttaaaaataagagcacatgagactatctttttatttttgtgaataaccaatttgaataagtaaacaggacaacctcagaataaattattttgaaaatacaagccttaaaaacacactattagctttttataaaaaagcgtttatagaaatactagataagattttgaaactattattacaaagactaaacaaagattaacaaaaatatttccataatctctacagatatactaaaaatgaaataatccaactagaattttacatattcaagtatggtataagaagtttaaaaaaacaaaagagttctaggaccagagtggctaaacataaaaagccacttataagattaaaatataaagcacaggctgggcgcggtggctcacgcctgtaatcccagcgctttgggaggcctaggcgggcggatcacgaggtcaggagatcaagaccatcctggctaacatggtgaaactccgtctctactaaaaatacaaaaaattagccggatgtggtggtgggcgcctgtagtcccagctacttgggaggctgaggcaggagaatggcgtgaacctgggaggtggagcttgcagttagccgagatcgcgccactgcactccagcctgggcgacagagcgagactctgtctcaaaaaataaaataaaataaaataaaaataaataaagcacaaaacattttaagagaaaaactaagaacttaattttaaaaattttccactataatacttaaagtaaatgctatttaagtcatattaacaattatttattaagcatactatgtagaaaaattggtggcccaggtctgtctgattcactatatgctgtcaaccagtgtaactagacaaaatgtgaaaactacaatgagaaaattacaagtaaaatgttatggtggtaatagagagggtgaggtatggagctgtccagcctgaattcaaatctcagctctaccacttcctagctgtatttggaaataatgacagtatacacttcaaaaacctatggtgaggataaacaatagaatacatgtaaaagactagcatagcacctggtacctagtaagcatctaacaaatgtcacttattttcatcattactgacagtctcataatctttatcttcatcctcaagcagtcacaggtgagatttttcatccctgtccacgtgtggacaagcagcttttgtttgcctggcatccattccccttcttctctttccaggtgcctcatttacttgggaaagcctgacctgtagtcctcagttctcacccctaggtgaggttcactctctaccctccagggtggacatgtggctcaagcctggccagaagttggttcagaaatgggcaaagggcctaagccaatcagaggcaactccaggatttttgtcaaagcccttggtaaagagcagctctctttctgtatgtatctccatgatgtaatgctgaaactattagggccatcttgttgtcatatcagggagcttacaagagaataactaatatagaaaagaaaagctgagagatcaagttttgataatattgtttgagcccttggttccaactctgccagtcaatcagcagacccataggtaaggaccaatacattttgttctggtttgcttaagctcatttgaaagatttttgttttgtttcggttatttgcatctgagaagctctataacacgtcaggtatcaatcaagagccagaagctggtgacctaaaatatagtagagcttagaagggcatggatttggaatcggagagatctagttcctaatcttgtttctaacaattactagctgcgtggccctgggcatgtgtctaatcctacttcttcatctgtaaaatggcttaatacctgcctcaaagggttgatgagaggataaatgacaatataaactgctcagtcttcttcataacaatgaacaatcaatagaggctattattatatcactgaatcccaagctgatctttgtatctgacctcttatattccatgtccctgtttccagtggtataaacagccatctttacctacatactctcctatccaacctcaaattcaacatttccaaatctcatcctgcctccatgcttcctcatcacaccctctgcctttccctttcagaaaaaaaaatgctcactggaggccaatttatcatcaattattaactatgttcccagtaggttttaatatcagaacagtaggagtcatcctagactgaaacttctttcctgttatctaatcagtcacaaagccctgttaaatatctgcagcctgacaggaaagaattttcctctccctgtggataaaaacaagggcaggattcttccaacttcaggatatgtagccttttcattggcttgcaaggagaactccaatccttctactcctttttctcatctcatcctccttgtggcttcatttcttttcatattcaggatagatttttgctagcttattttttcattaacctcaagctgcaaatcaacaaacgtagactaatccaatttattgcagttcccacacccaagctactagcaccactggaaaaagttataattatgcagattgacaccactaaaagttgtctctgacctcacctggaccttcagaactccttggcaatttctgtatttctggtcaggtttgttgatgttcctcaaaatggttatttcaaatctccattgctcacctctctcagccattattttagtcccaattttccttttttttttttttgagacggagtctcgctctgttgcccaggttggagtgcagtggcgcaatctcggctcactgcaagctccacctcccaggttcacgccattctcctgcctcagcctcccgagtagctgggactacaggcgcccgccaccacgcccgactaatttttttttttttttttgtatttttagtagagacggggtttcaccgtgttagccaggatggtctcgatttcctgacctcataatccacctgcctcagcctccccaagtgctgggattacatgcataagccaccacgcctggccagtcccaattttcttattcactagatggagatattttctatgtacttacctgagataattaaggcaaacaagcaaaaacttcctcaacttttcacccccagatttctaaatttatctaaataaataacccttgtcatctcatttccctcaatcagaatggctaggatgcgggaaagtacacaatatagtgatggaataaataaaaggaagagacattctttctcctatataaggccaatcctgcctcctatagtgcctctgaaattttaatatgcatacaaatcacctagggatagtgttaaaatgcatattctgattcaggaggtctggagtggggaataacgttcttcatgtcttccaacaaaccagtgttggtttgggagcaatatttagagaaaacagattccatcctttctcacctcctcagagatcttttactctaagtcaacatttgttgcttctttatcagctgctccctcccccaacatacaacatatctgtctcttttattttaaaataaatgctcctttgttcctcttttagctaccgcccaatcatctaggctctctttggctccacagacaagtttctatgaagaagtcctatgtcaacggttccaaactttcttggctcaaagaatctttagtgtctcagtgatcttgcagggtaaacctcggccaaaagaagtatctaacaactgtagttgcctccgttaagcagtaagagccaaaccacttagtaagtattttaagttctaaaaactggatgcctgttgggcactgcatacttttcaaactctggactcagattgaacactgacagcctcatttcctgttccacactgattttcaggcagtacttgctttttatcagagcaagcactgaaaatccagcttcccaaaggtatgacatcactgaaaggaatgcagtgatctaatacagcaatctaatgacgccagtgtatttcccttgaaatttaaaaatattccatggcacccttgcaagttagctacagtgctctgggatacatcgatgaacaatttgggatactgagttcaggtggctgtcagaacggcagcatccccattgcttgggaggttgttggaaatgtagaatctgggcatccatgccagacctactgattcagaatctccactttaacaagatctccaagtgatctcccagtgactcatagacacattaatgtagaccagaagtgctggtctaaggtccctgcttacgttttctcacataccactcacacccaacgcaccgctttctgactttgctcccacaactccagtgaaattaccccaggaaaagtcccaaattaccccactcctttaaaaagaaaaaaaaaaaaggcaataaatgtcttttagttccaatcctaattaaccctctctatagtgtttggtactattcatcttcttgaacctctattatttcttggtctctaaaacacccccgtcttctggttttcttccttttctttcaatacacatccccacccccttcctctactgacccaggcagtttttgttttttcctcactggcttctttttttttttttcttttttgagattggggagtctcgctctgttgccaggctggaatgcagtggcaggatctcggctcactgcaacctctgcctcctaggttcaagcgattctcctgcctcagcctcccaagcagctgggaccacaggcacgcgccaccaagcccagctaatttttgtacttttagtagagacggggtttcaccatgttggccaggatggtctcgatcttttggccttgagatccacctccctcagcctcccaaagtgctgggattacaggtgtgagccaccgagcccggcccctcattggcttctgtcatgagcaaatacccttgctatagacaacttttcctctggagaattacattcaatctcatgcccaccccagtaagtcgaggttgctctccagagccttaagtcatataaaaccaatttccatctggatgtccacaagcaaagcaaacttaacagttccaaagcaatttatttgtctgtcttccctggccttcacattactcttcttcatgtattatttcctttactgagtggcatccttgttcaaccagtctccctacggaaacttagaactatcatccttgactcatccttctccatttcccttgccctacacaatcaatcaaatcaaaccttcttgctatgtctctaatctgtataactctctctacctccaatacctacatcttaaaagaagctcttatcttttgcttcaattcctgttaacagtcttcttatctatctccctccctccagaattgccatgcccttctccaaactattctccacactccattcaagttagttttctaaaattaaaatctgatgtcactgccctcctctaaatccttccatagctcccaatgcctagcagtgcttctcaagcttatccaacaaatggcagaagagtttgtagttacagatctaagaggttcgctacaaacaagacatttcttgcaaggctgtttcattttaaatagtcatcttgattttatattctattccttgggtggccacttatgttattaaaagaatgttttaaatcaatataaatcaaactacttaactttttctccacaataactgagtaaaattacgttcaaggaagacacactggttacctgtggcttcaaatatcccttggcacaatgccactagaccagggattgacaaactatagcctgcaggccaaatatggcctaccactgatttttgtaaataaaattttactggaacacagtcaagctcaatctactgtctatggctactttcaaattacaatagcagagttgagtagttgcaacagagaccatgtagcccacagcctaaaatatttatgatctggccctttgggagtcacctgacccctgcatcaaaacatgaggattgttaaggctagaactgtgttttgtctttatgtcttcagcacagttctacagaaggtattcaaaaatatttaacttaataaattagaatgttgcaagaagatctccaagggcacataaggaaagtgtccacaaagctgatacagaatgcacctaaaccctaaggagaaatatacaggggccaaatagaaatgttccttcatcagtcagtgaaatctgaaaggctgagataagacagatgaaatggaatactactacacagaggtcaaagcaggttctgaagaaactgataagcccatcagacctaaagctacctccccaaagaaccacagtatataggactgtattgataacacatgcacctagtagcatctaagaaagagtcaggaactactattcagggcaaatgctatgagaattatttctggaatctttgtaattgcataatttgtacttctccaaatgatgagaaccaaaacacttaaaaccgaaatattttagatgacagtaggaaaatagggagctaaaagttcattctaccttgcagtcaatatctcaaaaagataattagttcaagaaatactagaaggaatggtaatttaaaaggaaagaaagaaagaaaggtaggaagggaagggtcagcaaactatggcctgtgggccaagtctagtccaacatctttttcttttttttaaagagatgggggtcacattatgttgtccaggctggctttgaactcctgggataaagcaattcttctgcctcagcctcccaagtagctgggactataggtgcataccaccatgcccagaccaccacccatttttgtaaataaagttttattggaacacagtcacacttacgtggtcacgtatttctatggctgctttcctgttacaacagcagagttaaacagttgcaacaaggacggcaaggctcacaaagcccaaactatctactatttgactctttggggtaaagaatctcctaatatccactctgggaagatggatctgataagagagaaagatgaatgtaaagaacagattggggaaaacaaagaataatggaggtagggagatgacattcacggctactgggggaaaaaatacacacacgataaagtgagggtttaactatagtagtgagaactgaagcaaaagatggatatttgagccttggtgtgagaacgcagagaggaaccagaaacagactcaaagacaatgaatgaatcaattcaatgctaaggtaccatgcctaaaaggagatcaagaacatgagtaaaaatatggaacacatgataaacagcaggtttcagaaggaagataatagggtaggattagtctgatattaagtgtgaaaactctaaaatgccaataagacatacagaaaacatttaatggcagtttgaaatacaccttaacagtgcaagaaggaaatctaactagagaaatagagttaggcatcaacagttcagtgacggtatctgaagatacagaagtggatgagatcaataagctagaaaagacagcaggagcagcagatcccattactcagcaactacagtgtttcagaccctatactgggcactttatattatctctaatcctcccaacaatcctgctataaaggaattacccctattttacagattagaaatctaagtttggaaaggattgaggaaatttaaaattccaaggtcatacagctaatatgtggcaggggtagaattcaaaccccagtctacctgacaggcctataatccttattacatatagagaagaatgttgacaccaaaaccagagagaaatactatttatagttagtgagaagaaaggaaaccagcaaagaaggaatggttcaagagatcacagaaagccatggcagtacagtatcatgtaagccaacagaggagaataaagccaacaggggagaggcgagtagaagcaggtggtaattttaatggctatgtgccaggtacagtgctttacatatattagctcatttacttctctcaataatcatgacataagtactattactatcccatgaaataaatatgagaaggcagataacttgctagcatcatacaaccagccaatggcagagctgggttttaaatctaggcaacccccctttgcatcctacattcttgaccaatatataccacgctacctgtgggcactacgaaatggttaaggagaaaaacacaggagagggaaaagggcagacccttttacaaggggaataacaactttcaaaagtttagttttagaacatcacttgaaggcacatgtcataaagaagttaacaaacaagcaactatcacataaagacagatagtatcatctacttttagaagccttggggaaaaaggtaagagaatattaatctatagggttaaaactatcaagaaaatataaaccaatttggggtattagaaggcctttttgctttgcaagcttataggttgtgagaggtgtatataagacttaataaatgtcctttgggtatctgagatataggatgtaactttttagtatcctttaacaatggagagtatgccctacacaagaattctcagacccaaatgtgaattagaatctccaggaaattgtctttaaattttttattttcgtgggaaaatagtaggtatatatatattttctccatggaattttaaggagctatgagtcttcacctcagattcagaatctctgattcagaagatctgtagtggggccctggcattaatgtgatcttgatccaaaacttatatacttacagaaactatgacaccctctgcatacattgcctgagttacttgaaaacagtatcttagtaaatgtttaaggttaatatgaactttactacttgaccaataccatatgttatgatttacatgaaaaaactaacctgctcaaaagatttgtatacgatatctgaatactatcaaaataatgttcctttgagtgaatggctgggtttttccttgagtgtgagtttgtacctgttgaaattaaacaacattccaatggtgcatttctgaaattacttcaataagggaaagactcatttaataaatatttggctcatcctaaccttttaaaattgagaggccatgtctggtggctcacgcctgtaatcccagcactttgggaagctgaggcgggtagattgcttgaggtcaggagttcaagaccagcctgggcaacatggtgaaaccccatctctacaaataatacaaaaattagccaggcatgctggtgcacatctggattcacagctactcaggagctgagatgggaggatcatttcagcccagaaggtggaagttgcaatgagctaagagatcgagcctctgcactccagcctgggcaacagagtgagaccctatctcaaaataaacaaataaataaacaagaaatttctgtaatgcatacagtgagatcaagtaggagacctgaaaataaatacaacacccctcaaaagatgcatagaaacttaacttcacatgtcttagctaggaagttaagctctagaatcaaactatcctggtttacctaactttctagctgtgtgatttggaacaagtttttaaatctctctgtgcctcagttacctcaattatagaatgaggaaaatagtaatgacctgcacctcatatggttgctatgataattaaatgaggtaatctgggtagaaagagcacctaatagtgcttggtacacaataagcattcaataaatgttggcaatataagcaaataaaaatgttcactagcccagcaattatagtctagaaatttttcttacaaaaaccttgttaaaatgtacagagatacatctttaagaatatctaatgcattgttggcatcagattatttgtgacagaaaaaaattacaaatagtacaaacttcaaccatgaagagctaaataatttatgttacaatttttaactcagcatgatgtttctgagattcatctgaagttgctgcataaaagactagttcacttcattttactgctaaatagtatctcattgtatgatgaagcacactctgtttactcaccagatgatggacatttgggttgtttcctatttttggtgataatgaataaaaatattcaagtctttgtatgaacgtgttttcacttctcttgatagatacttaggagtggaattcctgggtcatatgataaatttatacttaactttatgagaaatggctaacccgttttccaaagtggctgtaccattttgtatttccacaagtaatgaatgtatgaggattgcacatgttccatgtccttgccaaaacttggtatcatcaatatatttaattttagccattctagtgtatgtatagtggcatctcattgtggttttaatttttaatttttctggtaaccaatgatgttgagaatcttttcatgcacttggccattcctgtatcttcttttgtgaaaaagctgttcaatttttctgcctatttttcttttgggttgtttctcttcttcttattaatacattctttagacattctgatacaagtcttctgttgaatatgcttgtaaatattttctctcaaaagggtttgtcttttcattttcttaatggtatctttggataagcaaaacgttttaatgttgacatggtctaatttatcagtgtttttcttttatggctcatcttttttatgtcctaaaaaatatttgcttatcttgaggtcacaaggatttttctctgatgttttcttttaaagttttatggatttaggtcttactttttttttttttttgagatggagtctcactctgtcactgagactgaagtgcagtggtgcgatctcagctcattgcaacctctgcctcccaggttcaagccattctcctgcttcaccctcccgagtagctgggattacaggtgcacgccaccacatccaactaatttttgtatttttagtagagacagggtttcaccatgttggccaggctggtctggaacccctgacctcaagtgatctgccggcctcggcctcccaaagttctgggattacaagcatgagccactgcacacagcctaggtcttacatttaggtcagtgatccattttgaattaatttttataaatggtgggacataagggtcaaggtttattttttccacatagatagccatgtattccaaaaccatttacagaagaaaaaaaaaagaacaaaaacctagccagtactgaatttccttagtatctttattaaaaaaaaaaaatcaattgatattgttagtgtaggtctatttctgaactcttcaccctgtaccattgctctacatctatttcttctttgacccataggttatttagaaattcgctgtttaatttccacatatgagattttcaaaatttcattttattactgatttctaatttaatttcatagtggttagagaatatactctgtatgatttcaatccttaagttattaagacctgctttatggcctagcacctgttgtaccttggtaaatgtctcatatgtacttgcaaataacgtattttatgctgttaatgcaaggaatattctacagatgtcaatcaggtcaaattggttgatagtgttagaatcttttatatccttgttgatttcctatttaagaattactgagaaagaagtattgaaatttccagctataattgtaatttgttgattgtgcctttcatttctatcagattttgcttcatctactttggagcactgtattatttttataatcagaaactatataaagtcataaacaaagcagcagcctctttcccatgacatgtaagatttttacaggctgggtgcagtggctcatgcttgctatcccagaactttcagaggccaagggaggtggatcacctgaggttgggagttcaagaccagcctggctaacatggcgaaaccctgtctctactaaaaatacaaaaattagccaggcccagtggtgcgtgcctgtacttggcactcagctacttgggaggctgaggcaggagaatcgcttgaacctgggaagaggtggaggttgcagtgagccactgcactccagcatggtcaacagagccagactccatctcaaaaaaaaaaaaaaaaaaaaaaaaagatttttacagttaaaatatatatgcaattcccaaactgaaaagataacactacaagtatttaagctatattgttattaaattattcaaagtattgtataattcaaagtgtttacatttctacatacctagatattatatatttctataagaaaaacataggtatgaatacatgttgtttaacttcatttcctcaattccttattttcaaacaataagctttactatctttgtaaataactaattttatacaatgtcaacactaaaaaagctactccctattataatacatacagcagttcacatcttaatggtttcaagttttgactgttaaaaatgttaagaataattaattttagaaaaaagtgtggaacctacatgaaatttcttaaggacaacacaggctgcttaaaatacactttttactacgagcacaattactacatgagccaaatttgacagcacagacaccaaagtaattctacatattttataatggccacttaaaagacattattaagggtaaataagccataaataccccccattattttaggaggaaaataaataacttttattattttgtttttactgctacctattaagttatagactatacagtaaataaaagaaatactaagcaatatcaacaaactgtaaaacaaatcaaatatgggcttacgtcagtaagacctgtaaaataaggccaggcacagtggctcatgcctgtaatcccagctattcaggagggtgaggcaggaggattgcttgaggtcatgagttcaagaccatcctgggcaacatagcaagaccccgtctctaaaaaaaaaatttttttttaaagaataaactcatttgtgtgtgagatttgatccaatcattctagcttacctgcatccatcactagtttttaaaattaattaacatctaagggtttcaatttttttaatatagcttaaataattaaattcatgattataaataaaatattgccacttgacttcaatctgctaacggagaaggcatgaggagagaaagattttttaatcaaggtgttcctcagctggagttatttcattacccattaagtaacacaactatctgaaattcagttcaagatgcccttatcaactgaacactatttcctttctcacagtgccaaatctatgtgccaactgcctaagcaacaatagattcgctttaattaaaaataaggttctgacatcccagtaaaaccaagtcaagaattctgaatctaaaattgaatctgaacttctgatgacaatgttaaccaacaccaagaacagagttaagggcaactaagaccaagtcctaatctgaaaaggcactgctgagaaataaaactactttttattttatttatatttattatttttttgagacggagtcttgttctgtcgcccaggctggagtgcggtggcgctatctggcctcactgcaagctccgcctcccgggttcacgccattctcctgcctcagtctcctgagtagctgggactacaggctacaggtgcccgccaccatgcccggctttttttgtatttttagtagagacagggttttaccatgttagccaggatggtctcgatctcctgacctcgtgatccgcccacctcagcctcccaaagcgctgggattacaggtgtgagccaccatgcccagccaatataactacttttttaaaaagtcttttacaaataatcatctactcaacaactcttctgttaattaggtcagcattactcccatttcatagatggagagactggcagagttgttgagtgccttgctcagtagtagagagccagatgacaagaatccaaggcaatactagcaggtcctattcccattcctggcttctgtccacaaacatcttccccaccccacactactgttctccctttccccagacaattctaagaggaagaaacaccaagaatctctagagtaggcacacattaaagaaaaagcttacagctactatgcaacatcaaagcatagttcctgaaaattgtcaatacgaacaaattattcttccatattctgcagatactttctaccaaaactatccagagatgtttcagatctaaccattatagaaactattttaatataaaaaaactagtatgaattctagtttcattttagttagtaagcaccccccaccccccacccccacccaattccccagccgcggagcactactattactaaggctttttttttttttttttttaattcaggtaggctgaggcaggagaatggcatgaacccgggaggcggaacttgcagtgagccaagattgcaccactgcagtccagcctgggcgacagagccagagagactccgtctcaaaaaaaaaaaaaatttcaggttaatactactgaaatgccagttacaaggaaaaacaaaaatgcaaaacaaacgagaacagaggcagtgggagcaggggacaggacagggttaaaaggacaagaggaaaaagagaatcataagccagtgttaattattagtgataaagactctcagtcttttatggtacaactgcacatctagaattctaaagcaagaattaacctagcaaatgtgtctggagaggtatgccctccagagaagacattatcactatatggtagtttccatcctggtatataccggttaactttgttttgttctatagccagactgtataaggcatatatcttccagaagtccataggatatacataccattatttatgggggaaaagaaactcaaaccatagttctaaaggcagggagggagcaagaactgaacacacacagaaaccttattctaccataacgtgatatgaaatgcttaaaaatgtttagttttggtatggttttttggaactctaacggggtttttaacatagggtctatggaggggctttattacaagcagaacattgtgtgtactatgttcattttgagtgtgagaaagtgcaccgctcccactagattctgaaaggggtccagcagcaaaacaaagttgtgagacgctagcttatatgaccgccttggaaaaaacttctaagctatcggaaaagggtgttattttattatcacacatgacctttagcccaaaatttctttattcaagacattttattccaaaatactttgggttgtaaccacccaaaactacaaccactctttgcacataaatcatctgtggtaaatatttcttaggctactttccttaccaactctgtaatcagagaataaaaatacattttaagcatcaaaactaagcaaaactggcaggtcaaactgccatttttgttttaaggataaaaatgtttgaatgtgtgagaaaaccagattcaaatgacctttacactgccagatttctatttatctacactccaccatggccctcttttaatctagttaaagcaactgcttctcccacgttgattaaatccttgaatctagaaatgtggttaaggccaaaaggttttctcatcataaaagaaatatttctcaacttaattaactcatgccctcttttgttaaatacaactcttaaacctccttccacgtaattgacattttgagtaaattacagttcgaaaaccaaatcaaatccatgataattttagaagcttaaaattttgctcttgaaaattttactctgcacatcaacgtggtatcaatgttgaactttgcatctgctttctagctacaagcttttttcttctaggtcttataaaaagggtatataaagaagtcactttcaacattatgtctattatgctgttttgttttgaaggattcacacaccattggtaggggtgagggacataagatatgcaggccctctccattagattcatggatgtgtaaagttctattgcagttcataacctcatcaactgacttttggcctaagcaacgtcataaaacttcatttcacctttagagcaaacctttctcctagtacctggagttaaaaatagtgcaagccatgctgacctgggctatttttatctggaaaatagggactagaaacagtatagcaagcaaagaaaggcgagtttaccacaacaggattaaaactctcacacactttttaaaaagtcccactacagttttagaaatcggatggaaaggtggtaaagcagaggtgaggtacagggcagaggatcagctgagggcagttcactgagtgttagcaagatatgactaatactgtacgtaacaagcaataataaaagtcctctgccgaagacagaatagtataaaacctttccttcccctgcccttacagatttctagagaagataatttgggaataaaaggtataaaaggtttttgagaaaagagagatcaaaggacattcagagttcgggaggatatctgggaagcagacttagcttagatcctaaagggtgtagaagatctgcattgctggggaggagtgtgggagtggtaaaccagtcaaagaattcacagatgtgctgggaggaggagaaggggagcagaagcaggaaaacagtctggctgaaacactggttgtatttcaaagagcagtagcaaatcagggtggataaatggtgtgggcctaagcagctggtgagcattaaatgtgtaagaatcaaattgtatccttagcaaacctctgaagatttctgagtaagatttttatgagtgttggtaaatttatataggatgcatatgggtagtacatggagaaaaatgaattaaggggaaaaaaagtgtgtaacaatccagatagaaagtgatttttaaaattctaattagagcggtgcagtgggaattcaaatataatacttactgagtcctcgacgcaagatgctgagtacaccaatgttggaaatacaagggttctcgcctcaagtggctcactatagtagaggaaatgggaatggaaaccagccacagtgaagtatgctatcacggtgatgggaagtgccagaggaacagacaggacaaagttgtagctctgcttagagaaatgagggcaagaagcaagttagaggaccttctcaccactttccaaacgaaagaaatgaggaaatgcacagcactagaaaatgaaggtaggttcagggacctgcaaacaggttcctgtggctggaacagaaagtatatggtagtgaaatgttctttataaaaaagtagcaatggacagaatgaactagaaagatacaaaaagaaaaaaaacaaacaaacaggcttttgtaataatccaggacagcaatgaagagtgtaaatgggaatgaggaacaagagactaataggaaattagttttaaataagcaaaattttcaaactgataaaggaaagtatgttcttctaaaacatcatgctgtttccagacttggagactttcaatagacttaactccaacttaacctttagaagtcttagcctaatttcttaaaaaaaaaaaaaaacaaaaaaaaacctctcctggcctgcagaccaagttaggtctcctcttgtgtgatcccaataggatcctgtacttccatctcataaacatagcactgggaattacaggttcaacattgtatttccctcacacagttaagactccagaaaggcagagagaattactttgacctgggcactgtggtaccttcagtgcctaacacagatcagacactcagtaaatagctgttgaataactaaatgaagttcacaggatcaattttaaatctgttcctccatctcccccatcagacaggcttaccaatcctcattccttccaacaagaggaaacttcagtcttaatcacattgagtttgcaatacttccagaaaattcaggtagaaatgtctgtcagtcctctacagatgccaaagtaagagacagacgaaggaatctttaacaaatcctaatagctgaagtcatgattgttgttattactattatttgttacagttacagctaatactaattgagcactgattatctgttgagtatttctttattttctcacctgtcctcaaatagacctattggattaagtactgttattaactcctatcatactgatgaggaaattaatgctttaggttagaaaatgtgtctaaaatcagagagcaattaagtagcagaagagcttcaacacaggcaaactagtttaagaacttgtgcttccatgagcgcataagataacccagggggaaaaaaagtgtagactagagggaaaaaaagagaagacaaaccacagatcataaagaatagtaacatttaaggggcagacactgccaaggaagaactgacgtaagaactggtatttatattcttgtatgattcctcacacatatggaatggaaagtcacctaatgacttgcttcttgcaaaagaatacagcacaagtgatgggatgtcacttccatcatcagattacaagactgggacttcagtcttgccagcagacactattgcctttttggcttgcatactttattgaagaaagctgtcatgttagggaagcccacgtggcaagaaactgaagccctcagtccaacaacctgccaagaacttaatggtgccaataaccactgagtgaggctagaagcagatccctccccattcaagccttagaaggagaccacacaccctgaccaatgtcctgactgcagcctgtgagaaatagtgaggcagaagatgtagctagactgtgcccaattccttgtctacagaaagtatgacataacaaatgtgtgttgttttaagctgctaaactttggagtaatctgttatgtagcaataaatgactactacaaactaagaagaaaaatctagaaatatagatgttcaagttccttctacatacacatcttctgacagtcatgaacagaaagtgaactgaactttaggaaaaaaaactaccattgtaagttgaggaaaaaaagaaaactgaaaaatataagaaaaaacaactgaaaggataagacccaggctagtaaatgggtagccaaagattttaaaggtggggtggttaatagagctattgcagcagtaaaaatatctggtctaaggggtggggggcggggaatcactaaattttttagctaaaaagcaattttcaaggcatccatttcagagtagacctaacagaaattggtagaagacatcacacaggtaaaatatgccttgagatagaattaaagggtaattatataacaaaactatagaagccaagctcctagtcaccatttatagaggaaaattggaaatatcaatattaataagaaaaaattcagagagatggttaatatggtttggctctgtctcctcacccaaatctcatattgaattataatccccattgtcaggggagggacctggtgggaggtgactggatcatggtgacagtttccctcatgctgttctcatgatagtgagttctcatgagatcttatgttaaaagtgtttgacaattctccccgcctccttctgtcacgtaagacatgccttgcttcccttttgccttctgccataattgtaagtttcctgaggcctccccagccatgtggaactgtgagtcaattaaactgcttttctttataaattacccagtctcaggtcttcacagcagtgtgaaaacagacaaacacaatggtcaatctttttattccccaaacttatgattagtccttttcaatttacttcttagatttgacatttcctattaaggtgttattacataaaaattagccttaaactgaattgagatgagattacagcttcctgggttctggagttcatgtttcatgtttctggttttaatttttaaaaattatttcatacctgacaatctaatgtttacccatacttttgtgaccctctctcactttagaatcaaaagcaaggcttcctatgctcaaaagtcctggatagtaggccgggcacagtggctcatgcctgtaatcccagcactttgggaggcctaggtgggcggatcacgaggtcaggagttcaagatcagcccggccaacataatgaaaccctgtctctactaaacatacaaaaaaattagccaggtgtggtggtgggcacctgtagtcccagctactcaggaggctgagggaggagaatcacttgaacccagaaggcagaggttgcaggcagaggttgcagtgaaccaagaccacgccattgcactacagcctgggtgacagagtgagactccatctcaaaaaaaaaaaaaaaaaaaaaaaaaaaaaaaaaaaaaaggcctggatagtatattctaattttaatagtaccagtattattttatatttggtacttttatttgcaattccaatgccaggctccacatcaaagggatgaaaggctcatacaacactgatatcttcacagatcatccaatacagtccctaagctatcagtaactttcttcttcttcactactttaggaagctatcatatagtcatgtgtatcatacagtcatgtattgataagtcagtggaactatttttgatattttacggtaattccattcttcccaattttctgtaacagttttattttcaacagtaggtttaaatgaagatgttttatttctgacttcattttgacattgaatctaaatctatgcttcccttatgttgaaccattcaattttctctaatgttcccctaaagttcaaacttcgtatatttctttttacgtggattcacaagccatcttgaatgagtggttcatgcaaggaagcacagacatacttatagacaacactaaaagtgttgcaaggtttcctgcaataattcctgaaggcggttccactggtagagctgatacaagcatactgcacagagaaaccttaaaacctctagagaagaaaggatggggctagaaggtcaacagtagtagtagctagcacagccaatttgaaggaatgtgaaaatatagaacatataacttttttcttgttgcttggctgggaagtaagtgtcagcaagctatcataaatggcaacactgaaaaagtgggggcaagagaagaaaagcagaagacttaggtcaaaataaaacaataatttccttccaatcgctcaagccaaaaccctttaagtcattcatgactcttccattctttcataccccacatctaatgtatcagcaaatctcatttgattctactttcaaaatatatccagaatatatcctacacaaactgatctcatttcctatcctactacatcaatcacatatcctttactccttccactaaaaccacagtggtttccttgctgttttctgtttctttttttgagacagagtctcacgctgctgcccaggttggagtgcggtggcatcatcttggctcactgcaagctccacctcctgggttcaagtaattctcctgcctcagcctccagagtagctgggactacaggcggatgtcatcacgcctggctaatttttgtatttttagtagaggtgaggtttcaccatgttggccaggctggtctcgaactcctgacctcaagtgatccgcccacctcgacctcccaaagtgccgtgcttgctgttttctgaacacaccaaacacatttcaagttagggcctctgtatttgctatttaataccattcctgaaaatgctcctcttccacatatccacataactaatttcccacttcattcaagtcactatttaaatgtcaccttattttaccaatcttatctaaaagagtactccctagtcactctgtccttatcttattctccatcttaatatttaccacctaacgtgccatgtattacttgtacatctgtttattgactgtccctccccaccccacacttcctccaactagaagataaggtccatgagaaggggggctttttgttgttattcaggacaccctagctcctagaacagggcctgtatgtgcagggtactcaacatttgttgaatgaacaaatgagtgaaaatagtatcaaaacaggactcagaaaatataacaaagggtaaagagtacacgtattttgctttggtaaagaaatgggacctggaacactgaataagaaaaaagagaattccaaactcatgaaaactttatggggatctcaagtcaacagacactgtccaagctcttcagaaatgaaactaattctgaattggttatggaaaaatgtgattatgaagtacaaaggtggcagaaaagttgagttttgactacgaaataaggcatgaaataaactgttgtaagagaagattttggttaaccatctatatcaatttttttttagaagagatttagctgaaataaaaggcaaagacaagctctgaaaatggacttagtaaattatctgaatgccatcatgtagtcacagacaatgttccaagtgtggaaacgagggcacagtggaaaagaaggtggaatgtaagcacagaagtaaaatctaagaaaaagaaactgcccaagaatgtgctagaataaaagggagactgaaatagttggggtagctttaaaggcctaaagtgaaaaattaaacaaaaacaactaagctgacaacatcaatccaaaggcatatcagaaaggcacaggcttcagaaaacatggaagaagaacaaagatttcaagcctgatgggagagaaaagggaaaggaaaacaggagtaaactgtcactcagaaacacagagtgggaagacagctcctactaaagaaacaagagagggtaggcctacttcagggcaaacggctttaaatagtatcagaggaaagacagatggataaagaacatttaagaaaattaggtaggagaaaaatttaaactagaatgtcagataagccaacatgtagaatgacagaagcaaaagggagaagaaagtctcgaatgttcaagcatattctcaaaaatggcagaggcccagtgcgatggctcatacctgtaatcccagcacttttttttttttttttttgagacaggctctcgctgtcacccaggctggatgaagtgcagtggtgcaatcacagctcactgcaacctctacctcccaggctcaagcgatcctcccacctcagcccctcaagaagctgggactacagacatgtgccaccatgcctggttaatttttgtattgtttttagagatgggtttcaccacgttgccccctggtctggaactcctgggctcaagcaaatccgcccaccctggcctcccaaagtgctaggattacaggcatgagccagcactttgggagaccaaggcaggaggatcatttgaggccaacagttctagaccagcctagacaacataacgagatcccgtctctacaaaaaaatttaagaaattatccaggtgtggtgacacatgcctgtagtcccagctactacggagactcaagtgggaggattgtttgagcccaggagctggaggctgcagtaagccataattgcaccactgcactccagcctgggcatgttaggccctgtcctttaaaaaaaaaaaaaaaaaagcgtaaaatatgccaaaattagttaagtagaaatatgtataaagttacagatatgaatgaactaaatcaaaggtctgttgattttgtcaccagctctatgaccttgaagaagttaatctctctgggacttttttctcttaaaaaaatgttatttcttctcttttaaaaaaaaatgtgtaaatgcaggaaaagtggttaggtaaaaaaatgggtgatcattctaagcccttttttctccagtttatcggaggggaatgagacagggaaagcacagaattcttaaaagaatcagaaagatagcaaattttttaaatgggagaagtaatagttaagaacagttctgcatagcgctttaccatctaaaaggtgctttacatgaaacattatatgttcacctcacaacaagcctgagaagctaaaaacacaggtatcactgaacaccattttataaacggagcaactgacgactcaagaaattaagaaactggactaaggatacagaaaccatagcagacccaggatccaacccagatctaatggcagaaccaaatccattatctttcttttttttttttttgagacggagtctcgctctgtcgcccgggctggagtgcagtggcacaatcccggctcactgcaatctctgcctgccgggttcaagcgattctcctacctcagcctcccaagtagctgggactaaaaggcatgcaacactacacccagcttatttttgtatttttagtaaagacagggtttcaccatgttggttggccaggatggtctcaatccctcgaccttgtgatccacccgcctcagcctcccaaagtgctggggtaacaggcgtgagccaccgcacctggcccctttctttcttaataattttttttttaatagagatgaggtattgccatgttgcccaggctggtcttgaattcctggactcaagctatcgtcccacctcagcctcccagagtgttgggaattacaggggtgagttaccacatctggcccggaatccattatctttcaaagttaaaatgatctctattatcatctcctcctcctccacgtaattaaggatttgtgggtatttttgtggattattaggtagtagataagtaaagagttacatacatacaaaaaaaaaattttttttttttttttgagatagggttttgttctatcacctgggctcaagcaatcatcccacctgagaccatgggctcgcaccaccatgcctggctaattttttaatttttttagagacaaagtctcaccatttgcccaagccagtcttgaactcctgggctcaagcgatcctcctgcctcagcccctcaaaatgctgggattataggcgtgagccactaaagcccagcctaaaataatcttttaacgtaaaataaaattcctttcgaactaaaggagttagtttacatgtaaactacacatcaactcactacacaaaatgttttcacatatagttgatcacaatctattaagactaccctctcagtccacacgtcccttatagctgacagaattactgcagttgtactattttcagcctttctacatacagagtaactcacttttaacttaaattctgacatagttataaaatcattaaacaagtaacttcaatttgtgtatcctggatcccaagcatacaaagagaatggaaaaaggagataaaggtgtacttatgtatttaggtttaaataacataagtttattttctcacagttctacaagctgaaagtctgagatcaggatgccagcatggtctggtactgatgagggctctcttcctgggtcagtttcgtttttaacatctaagaggattgttcaccacatctctgttagtgataataaaacagctgcattttcctcatcctattctttgctctaaaagccataatcagccagacctaactaattaactacagtaatagtgaaaaatcttagtaatataatctgctgcttattagtcccaacctattttattactccttttctttatgtatgttttaacggaacagatgaagttttggattaaattccaaaaatgttatgtatatgattctagtatttctattttaatgggaaaatgttttccttcatagtggcgaagaaactccaacagaaaagttcaacattttcccatgatcctcactcctctgcattcctcttcctcctgtacccccaagtcaaggcacactactcttcacaaaacagcagaccatggtttctttctactgcatatcattgagactcttcttgatgaaaacaagcataagcaaggacataggatcaaagggcagggtaacggcaaaaaggagaaaatcccaaaaagaccagagaaagtggaaacaggagcttggttgtatcagtggctacaattacatttctgtttttctcctgtatgacttcatttttcccttatggttccttttttaaagtagttgcataaacagcggccagataagcatgtttgaagtagaaattcaggggaaaacaaagcctacactgaatgggccacagactatcatgtatcatctgattaccatgccctgacataaagagcatgcaatatacatgatgtaaataaaaaagaacatcagattcttagtcagaaaacctgaatttaagtcctaatatgctgcagaatttccaacaagtgaactaacttttcctgagtcccacttttctaatctataaaaaaagggatacaaccacctgcctactgctgtgaaagttgaaatgagaaaaatactacacatttgctgcatataacttttaaaatcactataatgttatgaaggataaaattctgaattgaagtctccctctgcaatcacataaaaaaaccatattctgacatctgttaacagtatactgacaggcgcataattcaggtggataaatacaaaagtggcttttaagccactatctgaaataaacattgtacagtcacctaataaaaagttttacacagcccaaccatagagtatgattactcacatataaagctagtgctattcacccactttcaacaaaatatatgtatcaaattgcagttccagcagtgtgctaaataatctacatctcatttaatgttctacctataactagcgtagttaaaaaagagcatgggtttgatatctgaatgacttaagtttgaacaccagccagggttatagtaagtggtagacctctgccaagtctgagctgaaaatccctcaaatgtaaattaggaataacactactcacttcacagaattaagattattgtggccaggtgaggtggttcaagtctgtaaccacagtactttgggaggacaaggcaggaggatcccctgagccgaggagttcgagatcaaatgatcagcaagaccctgactctacaaaaaaaattttttttcatttggccaagtatggtggtgtgtgcctacagtcccagctactcggaagctgagatggcaggatagcttgagtccaggaggtggaagctgcagtgggccatgcttacacccttgtactccagccagggcaacaaagcaagaccctgtctccaaaaaaaaaaaaaaaaatactctgattgtatctgtattgatcctaatatgatgcctaacacactgtggttggttgctcaatatacggtgggtggttattttgattgccttctcatttccacaaagcaaacacccttgttttttaggaataaattacatgttcaaacacgattaccaaaacctctttcagccaagaagtacaatgaaaggaattcctgaatatcatttatagatcctgaagttgcgtccctaaaattaatataagcagatcaaatccaaagagtttaaaggtcatccctccttcatcctgtcccttattacctgctaatatgtacagttacgtgacttaaaaccccaacaactagaactcttgaggaaggaggtacctatttcctaatagcatctctatgagaagcatttgctctaagagccaggctgtctgctgtcccttctctaaggacaggtgaaagcaggaagacaaaagagaaaagaggtctcagcttaacttggaaagctaaattaactaaggaaagcagatcaagaaagatttgggacatcatcttcttcaagaatccaatttaagcttatgtcccaactatatactttggtcagtcttcacagccctattgtctcaaactaatttctttcctgctctggagctcaggactttatgttcagatgtgacaatttcttaaagttgtgccctgacaactttcttaaaaattttatcaatgaaaatctagtgaaagaccaacgctgaaatttcatcagatcccccttttccttccagtttaatattattcaactgttcctttttaagactacaagaaattaagacttgtgccttcatccagcaatgaagtggcaataggtaaattagactccacattgtatttttccccacaaaatgttcacgtttataactttctcactgcctatgttcagtctttcgggcagcctgagaatcacatgtaatgactgtcaaagtagatgatctgacacacacatacagaaaaaccactagttagagtcagcgtgtgtgtacaaacatgcaactgtgatgctctcccaggtcaatctctcaaaacccacaaaatcagagactgatggtggaggagggtggcctgggagcagccacaaagaaaagatcaagatctgagaagtaaaatgaaaaagagctgcaagagagttggttaaggacgaaagagtctactgtttatcatgagggaggaaagagggaatagactaggtaagaagtgaagaaatctgtggaggaaaaaccgcaatagtaaagagaaaacaaaaagatctcaccaaattgggaggataaaacgagccctcctgcgccaattccgtgttgttgttttgttttgttttgttttccaggaagttacaaaacagttttcaacagaaggattacattaacaaagcaaataaaggaaactggtgctaaaaatgtagcctacctacactgagaggcctgaagacggaaatacttctttcccctttttgtaacgggtttcaaaacagtaggaacagggtgagggataagagctataacagggggaggggagtagccaagctcttcctgaatacagagtctttcgactaaatgcttattttaaatgtacctttttgcggtcagccggcaaattcctaactgaacacagcgtttgactctcaatcgaaatgtgatgagtggttcagacaccataggaaatttaacagcttatgcaaacacaaggcagatgcagaaccaggacttgctttgggtggtactctaacaccccttcactgggtggctaaaacgccactaaggtttgaattgtgaccaatagtggaatggaaaaaagaaaataggaaagttaaagcgcctacgttgagtcaaaaaaccatgcaattagccggagaagtttgtgaacgggcagtaagtggaaaacagtggatacaaaggggctaactagataaacagctactacaaattctgtccgtcttccttacggggagctacacaagaaagagcgagtcccttctatatagaggaatgctgcttctcagcctaggcagtttcagtcgcaggcaggggctgcccgccgagaagggcctgtccagcacaaacccgcagcacacacttac

SEQ ID NO: 4 - DNA, 46898 bp, Homo sapiens
  Intron (1)..(46898)
  Negative STRAND OF Intron (1)..(46898): snp rs479200 at 12964
gtaagtgtgtgctgcgggtttgtgctggacaggcccttctcggcgggcagcccctgcctgcgactgaaactgcctaggctgagaagcagcattcctctatatagaagggactcgctctttcttgtgtagctccccgtaaggaagacggacagaatttgtagtagctgtttatctagttagcccctttgtatccactgttttccacttactgcccgttcacaaacttctccggctaattgcatggttttttgactcaacgtaggcgctttaactttcctattttcttttttccattccactattggtcacaattcaaaccttagtggcgttttagccacccagtgaaggggtgttagagtaccacccaaagcaagtcctggttctgcatctgccttgtgtttgcataagctgttaaatttcctatggtgtctgaaccactcatcacatttcgattgagagtcaaacgctgtgttcagttaggaatttgccggctgaccgcaaaaaggtacatttaaaataagcatttagtcgaaagactctgtattcaggaagagcttggctactcccctccccctgttatagctcttatccctcaccctgttcctactgttttgaaacccgttacaaaaaggggaaagaagtatttccgtcttcaggcctctcagtgtaggtaggctacatttttagcaccagtttcctttatttgctttgttaatgtaatccttctgttgaaaactgttttgtaacttcctggaaaacaaaacaaaacaaaacaacaacacggaattggcgcaggagggctcgttttatcctcccaatttggtgagatctttttgttttctctttactattgcggtttttcctccacagatttcttcacttcttacctagtctattccctctttcctccctcatgataaacagtagactctttcgtccttaaccaactctcttgcagctctttttcattttacttctcagatcttgatcttttctttgtggctgctcccaggccaccctcctccaccatcagtctctgattttgtgggttttgagagattgacctgggagagcatcacagttgcatgtttgtacacacacgctgactctaactagtggtttttctgtatgtgtgtgtcagatcatctactttgacagtcattacatgtgattctcaggctgcccgaaagactgaacataggcagtgagaaagttataaacgtgaacattttgtggggaaaaatacaatgtggagtctaatttacctattgccacttcattgctggatgaaggcacaagtcttaatttcttgtagtcttaaaaaggaacagttgaataatattaaactggaaggaaaagggggatctgatgaaatttcagcgttggtctttcactagattttcattgataaaatttttaagaaagttgtcagggcacaactttaagaaattgtcacatctgaacataaagtcctgagctccagagcaggaaagaaattagtttgagacaatagggctgtgaagactgaccaaagtatatagttgggacataagcttaaattggattcttgaagaagatgatgtcccaaatctttcttgatctgctttccttagttaatttagctttccaagttaagctgagacctcttttctcttttgtcttcctgctttcacctgtccttagagaagggacagcagacagcctggctcttagagcaaatgcttctcatagagatgctattaggaaataggtacctccttcctcaagagttctagttgttggggttttaagtcacgtaactgtacatattagcaggtaataagggacaggatgaaggagggatgacctttaaactctttggatttgatctgcttatattaattttagggacgcaacttcaggatctataaatgatattcaggaattcctttcattgtacttcttggctgaaagaggttttggtaatcgtgtttgaacatgtaatttattcctaaaaaacaagggtgtttgctttgtggaaatgagaaggcaatcaaaataaccacccaccgtatattgagcaaccaaccacagtgtgttaggcatcatattaggatcaatacagatacaatcagagtatttttttttttttttttggagacagggtcttgctttgttgccctggctggagtacaagggtgtaagcatggcccactgcagcttccacctcctggactcaagctatcctgccatctcagcttccgagtagctgggactgtaggcacacaccaccatacttggccaaatgaaaaaaaattttttttgtagagtcagggtcttgctgatcatttgatctcgaactcctcggctcaggggatcctcctgccttgtcctcccaaagtactgtggttacagacttgaaccacctcacctggccacaataatcttaattctgtgaagtgagtagtgttattcctaatttacatttgagggattttcagctcagacttggcagaggtctaccacttactataaccctggctggtgttcaaacttaagtcattcagatatcaaacccatgctcttttttaactacgctagttataggtagaacattaaatgagatgtagattatttagcacactgctggaactgcaatttgatacatatattttgttgaaagtgggtgaatagcactagctttatatgtgagtaatcatactctatggttgggctgtgtaaaactttttattaggtgactgtacaatgtttatttcagatagtggcttaaaagccacttttgtatttatccacctgaattatgcgcctgtcagtatactgttaacagatgtcagaatatggtttttttatgtgattgcagagggagacttcaattcagaattttatccttcataacattatagtgattttaaaagttatatgcagcaaatgtgtagtatttttctcatttcaactttcacagcagtaggcaggtggttgtatccctttttttatagattagaaaagtgggactcaggaaaagttagttcacttgttggaaattctgcagcatattaggacttaaattcaggttttctgactaagaatctgatgttcttttttatttacatcatgtatattgcatgctctttatgtcagggcatggtaatcagatgatacatgatagtctgtggcccattcagtgtaggctttgttttcccctgaatttctacttcaaacatgcttatctggccgctgtttatgcaactactttaaaaaaggaaccataagggaaaaatgaagtcatacaggagaaaaacagaaatgtaattgtagccactgatacaaccaagctcctgtttccactttctctggtctttttgggattttctcctttttgccgttaccctgccctttgatcctatgtccttgcttatgcttgttttcatcaagaagagtctcaatgatatgcagtagaaagaaaccatggtctgctgttttgtgaagagtagtgtgccttgacttgggggtacaggaggaagaggaatgcagaggagtgaggatcatgggaaaatgttgaacttttctgttggagtttcttcgccactatgaaggaaaacattttcccattaaaatagaaatactagaatcatatacataacatttttggaatttaatccaaaacttcatctgttccgttaaaacatacataaagaaaaggagtaataaaataggttgggactaataagcagcagattatattactaagatttttcactattactgtagttaattagttaggtctggctgattatggcttttagagcaaagaataggatgaggaaaatgcagctgttttattatcactaacagagatgtggtgaacaatcctcttagatgttaaaaacgaaactgacccaggaagagagccctcatcagtaccagaccatgctggcatcctgatctcagactttcagcttgtagaactgtgagaaaataaacttatgttatttaaacctaaatacataagtacacctttatctcctttttccattctctttgtatgcttgggatccaggatacacaaattgaagttacttgtttaatgattttataactatgtcagaatttaagttaaaagtgagttactctgtatgtagaaaggctgaaaatagtacaactgcagtaattctgtcagctataagggacgtgtggactgagagggtagtcttaatagattgtgatcaactatatgtgaaaacattttgtgtagtgagttgatgtgtagtttacatgtaaactaactcctttagttcgaaaggaattttattttacgttaaaagattattttaggctgggctttagtggctcacgcctataatcccagcattttgaggggctgaggcaggaggatcgcttgagcccaggagttcaagactggcttgggcaaatggtgagactttgtctctaaaaaaattaaaaaattagccaggcatggtggtgcgagcccatggtctcaggtgggatgattgcttgagcccaggtgatagaacaaaaccctatctcaaaaaaaaaaaaaaaattttttttttgtatgtatgtaactctttacttatctactacctaataatccacaaaaatacccacaaatccttaattacgtggaggaggaggagatgataatagagatcattttaactttgaaagataatggattccgggccagatgtggtaactcacccctgtaattcccaacactctgggaggctgaggtgggacgatagcttgagtccaggaattcaagaccagcctgggcaacatggcaatacctcatctctattaaaaaaaaaattattaagaaagaaaggggccaggtgcggtggctcacgcctgttaccccagcactttgggaggctgaggcgggtggatcacaaggtcgagggattgagaccatcctggccaaccaacatggtgaaaccctgtctttactaaaaatacaaaaataagctgggtgtagtgttgcatgccttttagtcccagctacttgggaggctgaggtaggagaatcgcttgaacccggcaggcagagattgcagtgagccgggattgtgccactgcactccagcccgggcgacagagcgagactccgtctcaaaaaaaaaaaaaagaaagataatggatttggttctgccattagatctgggttggatcctgggtctgctatggtttctgtatccttagtccagtttcttaatttcttgagtcgtcagttgctccgtttataaaatggtgttcagtgatacctgtgtttttagcttctcaggcttgttgtgaggtgaacatataatgtttcatgtaaagcaccttttagatggtaaagcgctatgcagaactgttcttaactattacttctcccatttaaaaaatttgctatctttctgattcttttaagaattctgtgctttccctgtctcattcccctccgataaactggagaaaaaagggcttagaatgatcacccatttttttacctaaccacttttcctgcatttacacatttttttttaaaagagaagaaataacatttttttaagagaaaaaagtcccagagagattaacttcttcaaggtcatagagctggtgacaaaatcaacagacctttgatttagttcattcatatctgtaactttatacatatttctacttaactaattttggcatattttacgcttttttttttttttttttaaaggacagggcctaacatgcccaggctggagtgcagtggtgcaattatggcttactgcagcctccagctcctgggctcaaacaatcctcccacttgagtctccgtagtagctgggactacaggcatgtgtcaccacacctggataatttcttaaatttttttgtagagacgggatctcgttatgttgtctaggctggtctagaactgttggcctcaaatgatcctcctgccttggtctcccaaagtgctggctcatgcctgtaatcctagcactttgggaggccagggtgggcggatttgcttgagcccaggagttccagaccagggggcaacgtggtgaaacccatctctaaaaacaatacaaaaattaaccaggcatggtggcacatgtctgtagtcccagcttcttgaggggctgaggtgggaggatcgcttgagcctgggaggtagaggttgcagtgagctgtgattgcaccactgcacttcatccagcctgggtgacagcgagagcctgtctcaaaaaaaaaaaaaaaaaagtgctgggattacaggtatgagccatcgcactgggcctctgccatttttgagaatatgcttgaacattcgagactttcttctcccttttgcttctgtcattctacatgttggcttatctgacattctagtttaaatttttctcctacctaattttcttaaatgttctttatccatctgtctttcctctgatactatttaaagccgtttgccctgaagtaggcctaccctctcttgtttctttagtaggagctgtcttcccactctgtgtttctgagtgacagtttactcctgttttcctttcccttttctctcccatcaggcttgaaatctttgttcttcttccatgttttctgaagcctgtgcctttctgatatgcctttggattgatgttgtcagcttagttgtttttgtttaatttttcactttaggcctttaaagctaccccaactatttcagtctcccttttattctagcacattcttgggcagtttctttttcttagattttacttctgtgcttacattccaccttcttttccactgtgccctcgtttccacacttggaacattgtctgtgactacatgatggcattcagataatttactaagtccattttcagagcttgtctttgccttttatttcagctaaatctcttctaaaaaaaaattgatatagatggttaaccaaaatcttctcttacaacagtttatttcatgccttatttcgtagtcaaaactcaacttttctgccacctttgtacttcataatcacatttttccataaccaattcagaattagtttcatttctgaagagcttggacagtgtctgttgacttgagatccccataaagttttcatgagtttggaattctcttttttcttattcagtgttccaggtcccatttctttaccaaagcaaaatacgtgtactctttaccctttgttatattttctgagtcctgttttgatactattttcactcatttgttcattcaacaaatgttgagtaccctgcacatacaggccctgttctaggagctagggtgtcctgaataacaacaaaaagccccccttctcatggaccttatcttctagttggaggaagtgtggggtggggagggacagtcaataaacagatgtacaagtaatacatggcacgttaggtggtaaatattaagatggagaataagataaggacagagtgactagggagtactcttttagataagattggtaaaataaggtgacatttaaatagtgacttgaatgaagtgggaaattagttatgtggatatgtggaagaggagcattttcaggaatggtattaaatagcaaatacagaggccctaacttgaaatgtgtttggtgtgttcagaaaacagcaagcacggcactttgggaggtcgaggtgggcggatcacttgaggtcaggagttcgagaccagcctggccaacatggtgaaacctcacctctactaaaaatacaaaaattagccaggcgtgatgacatccgcctgtagtcccagctactctggaggctgaggcaggagaattacttgaacccaggaggtggagcttgcagtgagccaagatgatgccaccgcactccaacctgggcagcagcgtgagactctgtctcaaaaaaagaaacagaaaacagcaaggaaaccactgtggttttagtggaaggagtaaaggatatgtgattgatgtagtaggataggaaatgagatcagtttgtgtaggatatattctggatatattttgaaagtagaatcaaatgagatttgctgatacattagatgtggggtatgaaagaatggaagagtcatgaatgacttaaagggttttggcttgagcgattggaaggaaattattgttttattttgacctaagtcttctgcttttcttctcttgcccccactttttcagtgttgccatttatgatagcttgctgacacttacttcccagccaagcaacaagaaaaaagttatatgttctatattttcacattccttcaaattggctgtgctagctactactactgttgaccttctagccccatcctttcttctctagaggttttaaggtttctctgtgcagtatgcttgtatcagctctaccagtggaaccgccttcaggaattattgcaggaaaccttgcaacacttttagtgttgtctataagtatgtctgtgcttccttgcatgaaccactcattcaagatggcttgtgaatccacgtaaaaagaaatatacgaagtttgaactttaggggaacattagagaaaattgaatggttcaacataagggaagcatagatttagattcaatgtcaaaatgaagtcagaaataaaacatcttcatttaaacctactgttgaaaataaaactgttacagaaaattgggaagaatggaattaccgtaaaatatcaaaaatagttccactgacttatcaatacatgactgtatgatacacatgactatatgatagcttcctaaagtagtgaagaagaagaaagttactgatagcttagggactgtattggatgatctgtgaagatatcagtgttgtatgagcctttcatccctttgatgtggagcctggcattggaattgcaaataaaagtaccaaatataaaataatactggtactattaaaattagaatatactatccaggccttttttttttttttttttttttttttttttttttttttgagatggagtctcactctgtcacccaggctgtagtgcaatggcgtggtcttggttcactgcaacctctgcctgcaacctctgccttctgggttcaagtgattctcctccctcagcctcctgagtagctgggactacaggtgcccaccaccacacctggctaatttttttgtatgtttagtagagacagggtttcattatgttggccgggctgatcttgaactcctgacctcgtgatccgcccacctaggcctcccaaagtgctgggattacaggcatgagccactgtgcccggcctactatccaggacttttgagcataggaagccttgcttttgattctaaagtgagagagggtcacaaaagtatgggtaaacattagattgtcaggtatgaaataatttttaaaaattaaaaccagaaacatgaaacatgaactccagaacccaggaagctgtaatctcatctcaattcagtttaaggctaatttttatgtaataacaccttaataggaaatgtcaaatctaagaagtaaattgaaaaggactaatcataagtttggggaataaaaagattgaccattgtgtttgtctgttttcacactgctgtgaagacctgagactgggtaatttataaagaaaagcagtttaattgactcacagttccacatggctggggaggcctcaggaaacttacaattatggcagaaggcaaaagggaagcaaggcatgtcttacgtgacagaaggaggcggggagaattgtcaaacacttttaacataagatctcatgagaactcactatcatgagaacagcatgagggaaactgtcaccatgatccagtcacctcccaccaggtccctcccctgacaatggggattataattcaatatgagatttgggtgaggagacagagccaaaccatattaaccatctctctgaattttttcttattaatattgatatttccaattttcctctataaatggtgactaggagcttggcttctatagttttgttatataattaccctttaattctatctcaaggcatattttacctgtgtgatgtcttctaccaatttctgttaggtctactctgaaatggatgccttgaaaattgctttttagctaaaaaatttagtgattccccgccccccaccccttagaccagatatttttactgctgcaatagctctattaaccaccccacctttaaaatctttggctacccatttactagcctgggtcttatcctttcagttgttttttcttatatttttcagttttctttttttcctcaacttacaatggtagttttttttcctaaagttcagttcactttctgttcatgactgtcagaagatgtgtatgtagaaggaacttgaacatctatatttctagatttttcttcttagtttgtagtagtcatttattgctacataacagattactccaaagtttagcagcttaaaacaacacacatttgttatgtcatactttctgtagacaaggaattgggcacagtctagctacatcttctgcctcactatttctcacaggctgcagtcaggacattggtcagggtgtgtggtctccttctaaggcttgaatggggagggatctgcttctagcctcactcagtggttattggcaccattaagttcttggcaggttgttggactgagggcttcagtttcttgccacgtgggcttccctaacatgacagctttcttcaataaagtatgcaagccaaaaaggcaatagtgtctgctggcaagactgaagtcccagtcttgtaatctgatgatggaagtgacatcccatcacttgtgctgtattcttttgcaagaagcaagtcattaggtgactttccattccatatgtgtgaggaatcatacaagaatataaataccagttcttacgtcagttcttccttggcagtgtctgccccttaaatgttactattctttatgatctgtggtttgtcttctctttttttccctctagtctacactttttttccccctgggttatcttatgcgctcatggaagcacaagttcttaaactagtttgcctgtgttgaagctcttctgctacttaattgctctctgattttagacacattttctaacctaaagcattaatttcctcatcagtatgataggagttaataacagtacttaatccaataggtctatttgaggacaggtgagaaaataaagaaatactcaacagataatcagtgctcaattagtattagctgtaactgtaacaaataatagtaataacaacaatcatgacttcagctattaggatttgttaaagattccttcgtctgtctcttactttggcatctgtagaggactgacagacatttctacctgaattttctggaagtattgcaaactcaatgtgattaagactgaagtttcctcttgttggaaggaatgaggattggtaagcctgtctgatgggggagatggaggaacagatttaaaattgatcctgtgaacttcatttagttattcaacagctatttactgagtgtctgatctgtgttaggcactgaaggtaccacagtgcccaggtcaaagtaattctctctgcctttctggagtcttaactgtgtgagggaaatacaatgttgaacctgtaattcccagtgctatgtttatgagatggaagtacaggatcctattgggatcacacaagaggagacctaacttggtctgcaggccaggagaggtttttttttgtttttttttttttttaagaaattaggctaagacttctaaaggttaagttggagttaagtctattgaaagtctccaagtctggaaacagcatgatgttttagaagaacatactttcctttatcagtttgaaaattttgcttatttaaaactaatttcctattagtctcttgttcctcattcccatttacactcttcattgctgtcctggattattacaaaagcctgtttgtttgttttttttctttttgtatctttctagttcattctgtccattgctacttttttataaagaacatttcactaccatatactttctgttccagccacaggaacctgtttgcaggtccctgaacctaccttcattttctagtgctgtgcatttcctcatttctttcgtttggaaagtggtgagaaggtcctctaacttgcttcttgccctcatttctctaagcagagctacaactttgtcctgtctgttcctctggcacttcccatcaccgtgatagcatacttcactgtggctggtttccattcccatttcctctactatagtgagccacttgaggcgagaacccttgtatttccaacattggtgtactcagcatcttgcgtcgaggactcagtaagtattatatttgaattcccactgcaccgctctaattagaattttaaaaatcactttctatctggattgttacacactttttttccccttaattcatttttctccatgtactacccatatgcatcctatataaatttaccaacactcataaaaatcttactcagaaatcttcagaggtttgctaaggatacaatttgattcttacacatttaatgctcaccagctgcttaggcccacaccatttatccaccctgatttgctactgctctttgaaatacaaccagtgtttcagccagactgttttcctgcttctgctccccttctcctcctcccagcacatctgtgaattctttgactggtttaccactcccacactcctccccagcaatgcagatcttctacaccctttaggatctaagctaagtctgcttcccagatatcctcccgaactctgaatgtcctttgatctctcttttctcaaaaaccttttataccttttattcccaaattatcttctctagaaatctgtaagggcaggggaaggaaaggttttatactattctgtcttcggcagaggacttttattattgcttgttacgtacagtattagtcatatcttgctaacactcagtgaactgccctcagctgatcctctgccctgtacctcacctctgctttaccacctttccatccgatttctaaaactgtagtgggactttttaaaaagtgtgtgagagttttaatcctgttgtggtaaactcgcctttctttgcttgctatactgtttctagtccctattttccagataaaaatagcccaggtcagcatggcttgcactatttttaactccaggtactaggagaaaggtttgctctaaaggtgaaatgaagttttatgacgttgcttaggccaaaagtcagttgatgaggttatgaactgcaatagaactttacacatccatgaatctaatggagagggcctgcatatcttatgtccctcacccctaccaatggtgtgtgaatccttcaaaacaaaacagcataatagacataatgttgaaagtgacttctttatataccctttttataagacctagaagaaaaaagcttgtagctagaaagcagatgcaaagttcaacattgataccacgttgatgtgcagagtaaaattttcaagagcaaaattttaagcttctaaaattatcatggatttgatttggttttcgaactgtaatttactcaaaatgtcaattacgtggaaggaggtttaagagttgtatttaacaaaagagggcatgagttaattaagttgagaaatatttcttttatgatgagaaaaccttttggccttaaccacatttctagattcaaggatttaatcaacgtgggagaagcagttgctttaactagattaaaagagggccatggtggagtgtagataaatagaaatctggcagtgtaaaggtcatttgaatctggttttctcacacattcaaacatttttatccttaaaacaaaaatggcagtttgacctgccagttttgcttagttttgatgcttaaaatgtatttttattctctgattacagagttggtaaggaaagtagcctaagaaatatttaccacagatgatttatgtgcaaagagtggttgtagttttgggtggttacaacccaaagtattttggaataaaatgtcttgaataaagaaattttgggctaaaggtcatgtgtgataataaaataacacccttttccgatagcttagaagttttttccaaggcggtcatataagctagcgtctcacaactttgttttgctgctggacccctttcagaatctagtgggagcggtgcactttctcacactcaaaatgaacatagtacacacaatgttctgcttgtaataaagcccctccatagaccctatgttaaaaaccccgttagagttccaaaaaaccataccaaaactaaacatttttaagcatttcatatcacgttatggtagaataaggtttctgtgtgtgttcagttcttgctccctccctgcctttagaactatggtttgagtttcttttcccccataaataatggtatgtatatcctatggacttctggaagatatatgccttatacagtctggctatagaacaaaacaaagttaaccggtatataccaggatggaaactaccatatagtgataatgtcttctctggagggcatacctctccagacacatttgctaggttaattcttgctttagaattctagatgtgcagttgtaccataaaagactgagagtctttatcactaataattaacactggcttatgattctctttttcctcttgtccttttaaccctgtcctgtcccctgctcccactgcctctgttctcgtttgttttgcatttttgtttttccttgtaactggcatttcagtagtattaacctgaaattttttttttttttgagacggagtctctctggctctgtcgcccaggctggactgcagtggtgcaatcttggctcactgcaagttccgcctcccgggttcatgccattctcctgcctcagcctacctgaattaaaaaaaaaaaaaaaaaaagccttagtaatagtagtgctccgcggctggggaattgggtgggggtggggggtggggggtgcttactaactaaaatgaaactagaattcatactagtttttttatattaaaatagtttctataatggttagatctgaaacatctctggatagttttggtagaaagtatctgcagaatatggaagaataatttgttcgtattgacaattttcaggaactatgctttgatgttgcatagtagctgtaagctttttctttaatgtgtgcctactctagagattcttggtgtttcttcctcttagaattgtctggggaaagggagaacagtagtgtggggtggggaagatgtttgtggacagaagccaggaatgggaataggacctgctagtattgccttggattcttgtcatctggctctctactactgagcaaggcactcaacaactctgccagtctctccatctatgaaatgggagtaatgctgacctaattaacagaagagttgttgagtagatgattatttgtaaaagactttttaaaaaagtagttatattggctgggcatggtggctcacacctgtaatcccagcgctttgggaggctgaggtgggcggatcacgaggtcaggagatcgagaccatcctggctaacatggtaaaaccctgtctctactaaaaatacaaaaaaagccgggcatggtggcgggcacctgtagcctgtagtcccagctactcaggagactgaggcaggagaatggcgtgaacccgggaggcggagcttgcagtgaggccagatagcgccaccgcactccagcctgggcgacagaacaagactccgtctcaaaaaaataataaatataaataaaataaaaagtagttttatttctcagcagtgccttttcagattaggacttggtcttagttgcccttaactctgttcttggtgttggttaacattgtcatcagaagttcagattcaattttagattcagaattcttgacttggttttactgggatgtcagaaccttatttttaattaaagcgaatctattgttgcttaggcagttggcacatagatttggcactgtgagaaaggaaatagtgttcagttgataagggcatcttgaactgaatttcagatagttgtgttacttaatgggtaatgaaataactccagctgaggaacaccttgattaaaaaatctttctctcctcatgccttctccgttagcagattgaagtcaagtggcaatattttatttataatcatgaatttaattatttaagctatattaaaaaaattgaaacccttagatgttaattaattttaaaaactagtgatggatgcaggtaagctagaatgattggatcaaatctcacacacaaatgagtttattctttaaaaaaaaattttttttttagagacggggtcttgctatgttgcccaggatggtcttgaactcatgacctcaagcaatcctcctgcctcaccctcctgaatagctgggattacaggcatgagccactgtgcctggccttattttacaggtcttactgacgtaagcccatatttgatttgttttacagtttgttgatattgcttagtatttcttttatttactgtatagtctataacttaataggtagcagtaaaaacaaaataataaaagttatttattttcctcctaaaataatggggggtatttatggcttatttacccttaataatgtcttttaagtggccattataaaatatgtagaattactttggtgtctgtgctgtcaaatttggctcatgtagtaattgtgctcgtagtaaaaagtgtattttaagcagcctgtgttgtccttaagaaatttcatgtaggttccacacttttttctaaaattaattattcttaacatttttaacagtcaaaacttgaaaccattaagatgtgaactgctgtatgtattataatagggagtagcttttttagtgttgacattgtataaaattagttatttacaaagatagtaaagcttattgtttgaaaataaggaattgaggaaatgaagttaaacaacatgtattcatacctatgtttttcttatagaaatatataatatctaggtatgtagaaatgtaaacactttgaattatacaatactttgaataatttaataacaatatagcttaaatacttgtagtgttatcttttcagtttgggaattgcatatatattttaactgtaaaaatctttttttttttttttttttttttttgagatggagtctggctctgttgaccatgctggagtgcagtggctcactgcaacctccacctcttcccaggttcaagcgattctcctgcctcagcctcccaagtagctgagtgccaagtacaggcacgcaccactgggcctggctaatttttgtatttttagtagagacagggtttcgccatgttagccaggctggtcttgaactcccaacctcaggtgatccacctcccttggcctctgaaagttctgggatagcaagcatgagccactgcacccagcctgtaaaaatcttacatgtcatgggaaagaggctgctgctttgtttatgactttatatagtttctgattataaaaataatacagtgctccaaagtagatgaagcaaaatctgatagaaatgaaaggcacaatcaacaaattacaattatagctggaaatttcaatacttctttctcagtaattcttaaataggaaatcaacaaggatataaaagattctaacactatcaaccaatttgacctgattgacatctgtagaatattccttgcattaacagcataaaatacgttatttgcaagtacatatgagacatttaccaaggtacaacaggtgctaggccataaagcaggtcttaataacttaaggattgaaatcatacagagtatattctctaaccactatgaaattaaattagaaatcagtaataaaatgaaattttgaaaatctcatatgtggaaattaaacagcgaatttctaaataacctatgggtcaaagaagaaatagatgtagagcaatggtacagggtgaagagttcagaaatagacctacactaacaatatcaattgattttttttttttaataaagatactaaggaaattcagtactggctaggtttttgttcttttttttttcttctgtaaatggttttggaatacatggctatctatgtggaaaaaataaaccttgacccttatgtcccaccatttataaaaattaattcaaaatggatcactgacctaaatgtaagacctaggctgtgtgcagtggctcatgcttgtaatcccagaactttgggaggccgaggccggcagatcacttgaggtcaggggttccagaccagcctggccaacatggtgaaaccctgtctctactaaaaatacaaaaattagttggatgtggtggcgtgcacctgtaatcccagctactcgggagggtgaagcaggagaatggcttgaacctgggaggcagaggttgcaatgagctgagatcgcaccactgcacttcagtctcagtgacagagtgagactccatctcaaaaaaaaaaaaaaagtaagacctaaatccataaaactttaaaagaaaacatcagagaaaaatccttgtgacctcaagataagcaaatattttttaggacataaaaaagatgagccataaaagaaaaacactgataaattagaccatgtcaacattaaaacgttttgcttatccaaagataccattaagaaaatgaaaagacaaacccttttgagagaaaatatttacaagcatattcaacagaagacttgtatcagaatgtctaaagaatgtattaataagaagaagagaaacaacccaaaagaaaaataggcagaaaaattgaacagctttttcacaaaagaagatacaggaatggccaagtgcatgaaaagattctcaacatcattggttaccagaaaaattaaaaattaaaaccacaatgagatgccactatacatacactagaatggctaaaattaaatatattgatgataccaagttttggcaaggacatggaacatgtgcaatcctcatacattcattacttgtggaaatacaaaatggtacagccactttggaaaacgggttagccatttctcataaagttaagtataaatttatcatatgacccaggaattccactcctaagtatctatcaagagaagtgaaaacacgttcatacaaagacttgaatatttttattcattatcaccaaaaataggaaacaacccaaatgtccatcatctggtgagtaaacagagtgtgcttcatcatacaatgagatactatttagcagtaaaatgaagtgaactagtcttttatgcagcaacttcagatgaatctcagaaacatcatgctgagttaaaaattgtaacataaattatttagctcttcatggttgaagtttgtactatttgtaatttttttctgtcacaaataatctgatgccaacaatgcattagatattcttaaagatgtatctctgtacattttaacaaggtttttgtaagaaaaatttctagactataattgctgggctagtgaacatttttatttgcttatattgccaacatttattgaatgcttattgtgtaccaagcactattaggtgctctttctacccagattacctcatttaattatcatagcaaccatatgaggtgcaggtcattactattttcctcattctataattgaggtaactgaggcacagagagatttaaaaacttgttccaaatcacacagctagaaagttaggtaaaccaggatagtttgattctagagcttaacttcctagctaagacatgtgaagttaagtttctatgcatcttttgaggggtgttgtatttattttcaggtctcctacttgatctcactgtatgcattacagaaatttcttgtttatttatttgtttattttgagatagggtctcactctgttgcccaggctggagtgcagaggctcgatctcttagctcattgcaacttccaccttctgggctgaaatgatcctcccatctcagctcctgagtagctgtgaatccagatgtgcaccagcatgcctggctaatttttgtattatttgtagagatggggtttcaccatgttgcccaggctggtcttgaactcctgacctcaagcaatctacccgcctcagcttcccaaagtgctgggattacaggcgtgagccaccagacatggcctctcaattttaaaaggttaggatgagccaaatatttattaaatgagtctttcccttattgaagtaatttcagaaatgcaccattggaatgttgtttaatttcaacaggtacaaactcacactcaaggaaaaacccagccattcactcaaaggaacattattttgatagtattcagatatcgtatacaaatcttttgagcaggttagttttttcatgtaaatcataacatatggtattggtcaagtagtaaagttcatattaaccttaaacatttactaagatactgttttcaagtaactcaggcaatgtatgcagagggtgtcatagtttctgtaagtatataagttttggatcaagatcacattaatgccagggccccactacagatcttctgaatcagagattctgaatctgaggtgaagactcatagctccttaaaattccatggagaaaatatatatatacctactattttcccacgaaaataaaaaatttaaagacaatttcctggagattctaattcacatttgggtctgagaattcttgtgtagggcatactctccattgttaaaggatactaaaaagttacatcctatatctcagatacccaaaggacatttattaagtcttatatacacctctcacaacctataagcttgcaaagcaaaaaggccttctaataccccaaattggtttatattttcttgatagttttaaccctatagattaatattctcttacctttttccccaaggcttctaaaagtagatgatactatctgtctttatgtgatagttgcttgtttgttaacttctttatgacatgtgccttcaagtgatgttctaaaactaaacttttgaaagttgttattccccttgtaaaagggtctgcccttttccctctcctgtgtttttctccttaaccatttcgtagtgcccacaggtagcgtggtatatattggtcaagaatgtaggatgcaaaggggggttgcctagatttaaaacccagctctgccattggctggttgtatgatgctagcaagttatctgccttctcatatttatttcatgggatagtaatagtacttatgtcatgattattgagagaagtaaatgagctaatatatgtaaagcactgtacctggcacatagccattaaaattaccacctgcttctactcgcctctcccctgttggctttattctcctctgttggcttacatgatactgtactgccatggctttctgtgatctcttgaaccattccttctttgctggtttcctttcttctcactaactataaatagtatttctctctggttttggtgtcaacattcttctctatatgtaataaggattataggcctgtcaggtagactggggtttgaattctacccctgccacatattagctgtatgaccttggaattttaaatttcctcaatcctttccaaacttagatttctaatctgtaaaataggggtaattcctttatagcaggattgttgggaggattagagataatataaagtgcccagtatagggtctgaaacactgtagttgctgagtaatgggatctgctgctcctgctgtcttttctagcttattgatctcatccacttctgtatcttcagataccgtcactgaactgttgatgcctaactctatttctctagttagatttccttcttgcactgttaaggtgtatttcaaactgccattaaatgttttctgtatgtcttattggcattttagagttttcacacttaatatcagactaatcctaccctattatcttccttctgaaacctgctgtttatcatgtgttccatatttttactcatgttcttgatctccttttaggcatggtaccttagcattgaattgattcattcattgtctttgagtctgtttctggttcctctctgcgttctcacaccaaggctcaaatatccatcttttgcttcagttctcactactatagttaaaccctcactttatcgtgtgtgtattttttcccccagtagccgtgaatgtcatctccctacctccattattctttgttttccccaatctgttctttacattcatctttctctcttatcagatccatcttcccagagtggatattaggagattctttaccccaaagagtcaaatagtagatagtttgggctttgtgagccttgccgtccttgttgcaactgtttaactctgctgttgtaacaggaaagcagccatagaaatacgtgaccacgtaagtgtgactgtgttccaataaaactttatttacaaaaatgggtggtggtctgggcatggtggtatgcacctatagtcccagctacttgggaggctgaggcagaagaattgctttatcccaggagttcaaagccagcctggacaacataatgtgacccccatctctttaaaaaaaagaaaaagatgttggactagacttggcccacaggccatagtttgctgacccttcccttcctacctttctttctttctttccttttaaattaccattccttctagtatttcttgaactaattatctttttgagatattgactgcaaggtagaatgaacttttagctccctattttcctactgtcatctaaaatatttcggttttaagtgttttggttctcatcatttggagaagtacaaattatgcaattacaaagattccagaaataattctcatagcatttgccctgaatagtagttcctgactctttcttagatgctactaggtgcatgtgttatcaatacagtcctatatactgtggttctttggggaggtagctttaggtctgatgggcttatcagtttcttcagaacctgctttgacctctgtgtagtagtattccatttcatctgtcttatctcagcctttcagatttcactgactgatgaaggaacatttctatttggcccctgtatatttctccttagggtttaggtgcattctgtatcagctttgtggacactttccttatgtgcccttggagatcttcttgcaacattctaatttattaagttaaatatttttgaataccttctgtagaactgtgctgaagacataaagacaaaacacagttctagccttaacaatcctcatgttttgatgcaggggtcaggtgactcccaaagggccagatcataaatattttaggctgtgggctacatggtctctgttgcaactactcaactctgctattgtaatttgaaagtagccatagacagtagattgagcttgactgtgttccagtaaaattttatttacaaaaatcagtggtaggccatatttggcctgcaggctatagtttgtcaatccctggtctagtggcattgtgccaagggatatttgaagccacaggtaaccagtgtgtcttccttgaacgtaattttactcagttattgtggagaaaaagttaagtagtttgatttatattgatttaaaacattcttttaataacataagtggccacccaaggaatagaatataaaatcaagatgactatttaaaatgaaacagccttgcaagaaatgtcttgtttgtagcgaacctcttagatctgtaactacaaactcttctgccatttgttggataagcttgagaagcactgctaggcattgggagctatggaaggatttagaggagggcagtgacatcagattttaattttagaaaactaacttgaatggagtgtggagaatagtttggagaagggcatggcaattctggagggagggagatagataagaagactgttaacaggaattgaagcaaaagataagagcttcttttaagatgtaggtattggaggtagagagagttatacagattagagacatagcaagaaggtttgatttgattgattgtgtagggcaagggaaatggagaaggatgagtcaaggatgatagttctaagtttccgtagggagactggttgaacaaggatgccactcagtaaaggaaataatacatgaagaagagtaatgtgaaggccagggaagacagacaaataaattgctttggaactgttaagtttgctttgcttgtggacatccagatggaaattggttttatatgacttaaggctctggagagcaacctcgacttactggggtgggcatgagattgaatgtaattctccagaggaaaagttgtctatagcaagggtatttgctcatgacagaagccaatgaggggccgggctcggtggctcacacctgtaatcccagcactttgggaggctgagggaggtggatctcaaggccaaaagatcgagaccatcctggccaacatggtgaaaccccgtctctactaaaagtacaaaaattagctgggcttggtggcgcgtgcctgtggtcccagctgcttgggaggctgaggcaggagaatcgcttgaacctaggaggcagaggttgcagtgagccgagatcctgccactgcattccagcctggcaacagagcgagactccccaatctcaaaaaagaaaaaaaaaaaaagaagccagtgaggaaaaaacaaaaactgcctgggtcagtagaggaagggggtggggatgtgtattgaaagaaaaggaagaaaaccagaagacgggggtgttttagagaccaagaaataatagaggttcaagaagatgaatagtaccaaacactatagagagggttaattaggattggaactaaaagacatttattgccttttttttttttctttttaaaggagtggggtaatttgggacttttcctggggtaatttcactggagttgtgggagcaaagtcagaaagcggtgcgttgggtgtgagtggtatgtgagaaaacgtaagcagggaccttagaccagcacttctggtctacattaatgtgtctatgagtcactgggagatcacttggagatcttgttaaagtggagattctgaatcagtaggtctggcatggatgcccagattctacatttccaacaacctcccaagcaatggggatgctgccgttctgacagccacctgaactcagtatcccaaattgttcatcgatgtatcccagagcactgtagctaacttgcaagggtgccatggaatatttttaaatttcaagggaaatacactggcgtcattagattgctgtattagatcactgcattcctttcagtgatgtcatacctttgggaagctggattttcagtgcttgctctgataaaaagcaagtactgcctgaaaatcagtgtggaacaggaaatgaggctgtcagtgttcaatctgagtccagagtttgaaaagtatgcagtgcccaacaggcatccagtttttagaacttaaaatacttactaagtggtttggctcttactgcttaacggaggcaactacagttgttagatacttcttttggccgaggtttaccctgcaagatcactgagacactaaagattctttgagccaagaaagtttggaaccgttgacataggacttcttcatagaaacttgtctgtggagccaaagagagcctagatgattgggcggtagctaaaagaggaacaaaggagcatttattttaaaataaaagagacagatatgttgtatgttgggggagggagcagctgataaagaagcaacaaatgttgacttagagtaaaagatctctgaggaggtgagaaaggatggaatctgttttctctaaatattgctcccaaaccaacactggtttgttggaagacatgaagaacgttattccccactccagacctcctgaatcagaatatgcattttaacactatccctaggtgatttgtatgcatattaaaatttcagaggcactataggaggcaggattggccttatataggagaaagaatgtctcttccttttatttattccatcactatattgtgtactttcccgcatcctagccattctgattgagggaaatgagatgacaagggttatttatttagataaatttagaaatctgggggtgaaaagttgaggaagtttttgcttgtttgccttaattatctcaggtaagtacatagaaaatatctccatctagtgaataagaaaattgggactggccaggcgtggtggcttatgcatgtaatcccagcacttggggaggctgaggcaggtggattatgaggtcaggaaatcgagaccatcctggctaacacggtgaaaccccgtctctactaaaaatacaaaaaaaaaaaaaaaaattagtcgggcgtggtggcgggcgcctgtagtcccagctactcgggaggctgaggcaggagaatggcgtgaacctgggaggtggagcttgcagtgagccgagattgcgccactgcactccaacctgggcaacagagcgagactccgtctcaaaaaaaaaaaaaaggaaaattgggactaaaataatggctgagagaggtgagcaatggagatttgaaataaccattttgaggaacatcaacaaacctgaccagaaatacagaaattgccaaggagttctgaaggtccaggtgaggtcagagacaacttttagtggtgtcaatctgcataattataactttttccagtggtgctagtagcttgggtgtgggaactgcaataaattggattagtctacgtttgttgatttgcagcttgaggttaatgaaaaaataagctagcaaaaatctatcctgaatatgaaaagaaatgaagccacaaggaggatgagatgagaaaaaggagtagaaggattggagttctccttgcaagccaatgaaaaggctacatatcctgaagttggaagaatcctgcccttgtttttatccacagggagaggaaaattctttcctgtcaggctgcagatatttaacagggctttgtgactgattagataacaggaaagaagtttcagtctaggatgactcctactgttctgatattaaaacctactgggaacatagttaataattgatgataaattggcctccagtgagcatttttttttctgaaagggaaaggcagagggtgtgatgaggaagcatggaggcaggatgagatttggaaatgttgaatttgaggttggataggagagtatgtaggtaaagatggctgtttataccactggaaacagggacatggaatataagaggtcagatacaaagatcagcttgggattcagtgatataataatagcctctattgattgttcattgttatgaagaagactgagcagtttatattgtcatttatcctctcatcaaccctttgaggcaggtattaagccattttacagatgaagaagtaggattagacacatgcccagggccacgcagctagtaattgttagaaacaagattaggaactagatctctccgattccaaatccatgcccttctaagctctactatattttaggtcaccagcttctggctcttgattgatacctgacgtgttatagagcttctcagatgcaaataaccgaaacaaaacaaaaatctttcaaatgagcttaagcaaaccagaacaaaatgtattggtccttacctatgggtctgctgattgactggcagagttggaaccaagggctcaaacaatattatcaaaacttgatctctcagcttttcttttctatattagttattctcttgtaagctccctgatatgacaacaagatggccctaatagtttcagcattacatcatggagatacatacagaaagagagctgctctttaccaagggctttgacaaaaatcctggagttgcctctgattggcttaggccctttgcccatttctgaaccaacttctggccaggcttgagccacatgtccaccctggagggtagagagtgaacctcacctaggggtgagaactgaggactacaggtcaggctttcccaagtaaatgaggcacctggaaagagaagaaggggaatggatgccaggcaaacaaaagctgcttgtccacacgtggacagggatgaaaaatctcacctgtgactgcttgaggatgaagataaagattatgagactgtcagtaatgatgaaaataagtgacatttgttagatgcttactaggtaccaggtgctatgctagtcttttacatgtattctattgtttatcctcaccataggtttttgaagtgtatactgtcattatttccaaatacagctaggaagtggtagagctgagatttgaattcaggctggacagctccatacctcaccctctctattaccaccataacattttacttgtaattttctcattgtagttttcacattttgtctagttacactggttgacagcatatagtgaatcagacagacctgggccaccaatttttctacatagtatgcttaataaataattgttaatatgacttaaatagcatttactttaagtattatagtggaaaatttttaaaattaagttcttagtttttctcttaaaatgttttgtgctttatttatttttattttattttattttattttttgagacagagtctcgctctgtcgcccaggctggagtgcagtggcgcgatctcggctaactgcaagctccacctcccaggttcacgccattctcctgcctcagcctcccaagtagctgggactacaggcgcccaccaccacatccggctaattttttgtatttttagtagagacggagtttcaccatgttagccaggatggtcttgatctcctgacctcgtgatccgcccgcctaggcctcccaaagcgctgggattacaggcgtgagccaccgcgcccagcctgtgctttatattttaatcttataagtggctttttatgtttagccactctggtcctagaactcttttgtttttttaaacttcttataccatacttgaatatgtaaaattctagttggattatttcatttttagtatatctgtagagattatggaaatatttttgttaatctttgtttagtctttgtaataatagtttcaaaatcttatctagtatttctataaacgcttttttataaaaagctaatagtgtgtttttaaggcttgtattttcaaaataatttattctgaggttgtcctgtttacttattcaaattggttattcacaaaaataaaaagatagtctcatgtgctcttatttttaagatgcgtattttaagcattaaaaccatatgttttgtaacagagtttgagtttttagactaataaaagttaccgagtaccattttattttggttttcatgatttggggctctgcggtgccaattatatttttttaatcctgtctgaaatgtctgatctgtgtcgtcataaaggaagcagtgcaggattggaaatcaggagacccctttttgtcctttgggattcccctctctaaagagtttgggccagacctttatgatcttttccacttttaaaattctagtgtttttattttaaatctctgctagatcagaagcactgccttatggatgtatagtcttagagctagattggaagatagcatagtccaggttccttattttaaagatataccagctgggcatggtggctcacacctgtaatcccaggacttggggaggccaagatgggaggatcacttgagcccatgagtttgagaccagcctgggcaacagaatgagttccctgtctcacacacacacacacacacacacacacacacacacacacacaaaaaaaaaacagaatgagttccctgtctcacacacacacgcgtcggtcgtggtgaatcagttcctgtctacacgcacactagccgggcatggtggcttgcgtctgtagtcccagctactcaggagactggggcaggagtattgcttgagctcaggagtttgacgttgcggtgagctatgatcacgccactgatctccagcctgggggacagagaccttgtctcttaaaaaaaaaaaaaattaataatagtaaagataatccaggcccaaatgatagcgtctaagatctcaccatcatttagaagtagtggagcaaggacaggcatttctgcctgtgctgccttatgctcttctttcctcaaaacggtattagcaacttgaagacagacaacattcatttatttgattaaaggaacatttattgaatcgcttcaatctatcaggcattttactaggcatgtggttagctgcaaagatgtgtacatgagggaatgtgcatatatggaggcccaaaactggaatatagagtccaacaaccttgggttcacagtctatctgaccacttaggagcatgacctcagttaagttagccattctaagccgcatttctttgtctacaaaatgagaataatagtagtagacatttacacagttgtggtgaggattgagactataaagattaaacttagcatagtacgtggcacatagtagactctcttagagtttatgaaagaaggaaggaattaagacttatggaacattttcctgtattgtcttatttgattcatacccctgtgaggtagatagtgataccctaatttttcaagtgaggaaacagcgctgaaggcttgggacttccccaaggtccacagccattagtcatacctaccatacacattatttttaaaaagtgcaccagacactgttttaagcatttttatgtactagtttattagctttgcatgtggtatactattgttattttcacttaacagacaagggtagttaggtacagagaagttgaataatttgcccaaagtaacacagtcagtaagtggcagagtcgtgtgcttcatgtgcaggaaatctggcttcagagtcccactgttcttttgttgttgttgttgtttgtttgtttgtttgttttcagagagcatcttgctgtattgccaggctggagtacagtggcgctatctcagttcactgcaacctctgcctcccgggttcaagtgatcctcctgcctcagcctcccaagtagctggggctacaggcgcttgccaccacacccagctgattttttgtatttgtagtagagatggggtttcaccatgttgggcaggatggtctcaatctcttgacctcgtgacccgcctgcctcggccccccaaagtgctgggattacaggcgtgagccaccgcacccggtcagagccccactgttcttaatctttgtgttcttccttttacttcatgctctcttttgtgaaggtgaaaagcagttttacaaactagaaggtgctacataagatgtggtatatttatctgacttggtccctgtactccagagattacagtccatgaggaatgtgtcttctccaggctaaacctactcatttattgccactgttactcgctggatattgctgtcactattttggccacttttctttaaaaaaaaattttttttcattttgtcaacgttcgccctctaatgtgtccccagaaatggatacaagcttcaaagatctctattctgccaagctgtcttaccctcattttatagtttactctgcaagatgagaataaaacctgccttccagggattgtaaagattaagtgaaagaataaatgtaaagttattaacacatagtaatttgtcagtaaatgctgctggttattacttctgtatagtatggttgatttttgccattttgaaagtaggatttatttttaactttattagaattcatcatgttgattcaattgagatcacattgaatcttctttcttttatttgtgatattggctacttttcccagctttgtatgagctgtatattttcctattgagaaaggttacattaataagattcctttatttgtgtgactcaaaatcattgataaaaatatagactagctaggctgggcgcggtggctcacgcctgtaatcctagcattttgggaggctgaggtgggcagatcgcctgaggttgggagtttgagaccagtctggccaacatggtgaaatcctgtctctattaaaaatataaaaattagctgggcgtggtggtgggcgcctgtagtcccagctgcttgggaggctgaggcaggagaatcgcttgaactcaggaggcggaggttgaagtgagctgagatcacgccattgcacttcagcctgggcgacagagagagacgccgtctcaaaacaacaacaacaacaacaaaaaaccaaaaaattaggtaggcatggtggtgcatgcctgtaatcccagctacttgggaggctgcggcaggggaattgtttgaaccagggaggtagaggttgcagtagccaagatcacgccactgccctccagcctgggcgacagagtgagactgagacagacagagagcgagacagagacagacactagctacaggctagagatagtagaacctcctggcaggctggcagagaggcctggatgtaggattcatagagaaactaactattaatgttagtttgcccagctcacatttcaccgtcttatcaaccacatatgtgagaatatttttcaagattttaaaaaatgtatctagttggttcttccctcttaattttcttacagttctctcaatttaattacaaatgtgaggcctccatcctctgtccttagacttcttgcaacattctctttaaattagtcatatcctttagcagtggtactcaatttttgttttatggcccatcaatcactttgcttagagtcactgggttctaacaccctctacaaccaaaataaacttcctcttttgtcaacaagacatttagatgtaatagtgtatagcaatctgtaaagatctgcctacagtagatttacatacaatgatattgatcagatgagcagggtagatcacttaacaggttcagcaaacattgtgacataaagattaagcctgggccaccagaaaactgtggatcaaagaaacaggtgactgaagaataggtgagaggcagaagagtggtatagatggatgttctggtgggtggtgggaacaagcatttgagagagagattggatattggtagttactgacagtatgcctgacagatattgatgggatgtaaggatatagagaacttgacttcttaatagttgttatgaaaatcttgtactcagtctgtttttctaaaccactcactaggtatacttaaacatccttaaaaataagttgcggccgggtgtggtggctcacgcctataatcccagcactttgggaggccgaggcgggcagatcatgaggtcaggagatcgagaccatcctggctaacacggcgaaaccccgtctctactaaaaatacaaaaaattagccggacgtggtggcaggcacctgtagtcccagctactcgggaggctgaggcaggagaatggcatgaacccgggaggtggagcttgcagtgagccgagatcaagccactgcactccagcctgggcgacagagcgagacgccgtcacaaaaaaaaaaaaaaaaattaaaaaaataggttgcatctagggccaaaataaaaacacatggtactgaatgaaaaccatctgtaggcataatttagtattctgtctttatttaattaggggggtgtgtgtgtgtgtgtgtgtatacatatatagcatgccggtgttattttgacatctcatgcaaatggcaagttgtcaaacttcaatttttttttttttgactagccacaggggctcacacctgtagtctcagtgctttgggaggctgaggcaggaggattgcgtgaggccaggagtttgagaccaccctgggcaacatagcgagacctcatctgtacaaaacattttaaaaaattagctgggtgtgatggtgcacacctgtagagctgttcaggaggctgaggcaggaggattactcccactcaggagtttgaggttgtagtgagctatgatccaagccactgcgccctagcctaagtgacagaatgagaccttgtgtctaaaatatatgtatatatatttttacaagctagcagtacttctatcaatctttagcaatggatggtaaaacccaccttctgttttaaaattgtattaaagatgtttatgttactcttctttatctgtctaatctatcatctctgttctctcaaggtaaatcattaattgatttattcagtagatatttgagtgcctgtgtttggcactaggagtacaagaataaataagagctggttcatcctctgttggagtctacagtctggttggggaaacccagaaaaaaaaatgagtgtgtttaggtgtggcaatatataaataattaagtaacttttctaagaatgatgtgaaaaaacaaataacattaaactaattcaaagtgataaaagtagaatgaggttaatgttagatgaaacagactcatcattattttctaagactggatcttgtccttacattaaccacactctcccgcagtggtatggatgcaaagattaaacacttcagctcttaaaacaagactagataaaattatttgtaaaatggagatagtcatttctctgaatacctcatggagttgttgcagaattgaaatgagataattatttgtaaatgctgtgaactagagttctatacaaatagaacttgtactctgatgaaagttgtgacttgaatctttgtccatcatgccacaaaaaaacttttcagaagtgtttggttatctgcctacaaagggagttacgctattattgaaaaaaaaaaccagccgggcatggtggttcgtgcctgtaattccagcactttgggaggccaaggcaggcagatcacttgagtccagaagtccgaggccagctgggcaacatagtaagtaagaccctgtctctataaaaaatttaaaaattagctagtctactcaggaggctgaggcaggagcccaggaggctgaggctgcattgagccatgatcacgccactgcactccagcaacagagcaagaccgtgtctcaggaaaacaaaaatagaaaaagaaaaaaatcagctcctttttattgcagttactgaattcttaagagcctgatttacatgttttctggccagtcttagcaacttactgctttatagctgtactacaaaaatggtgccactcagtcacctaccgaagtgaatgaaaacctctagtctgaatgtgtcacttctttgtttaaacctcttcagtggctccctgttacccactaaaacccaagttcatcattggggcttactaagcttttgggaaggggccagagctatgtccagccttacctgtcacattccttgactcacacgtcaggttcacccttgtttgcagtttacacagcatgtgtctacacacacaccatgaccttttctcttcctggaatatgccccctctccttccttacctggctaattcatactctttcaaaactcatttttcagatagtccttcctgatcccaaccttctccccagtgaagtagtcagcgcatggactggtcattttaattgtcatctgtgttggaatgatcagttttcctctctgttctactcacacttcctagcactatgccttacacataccctaactgaacatgttcaaaactgaactcctgatctctcccattgtccagactcaggctgcaccatatgctgtcttcatctgtttatggtaattacattttccctattgcttaaaccaaaaccttagagtcatcttcttttatatctcataccagtctttctggacatcttgctttattctgcctttaagatagtatgagtatcccagcacttcttaccacctccattgctaccactgtggtccaagtgatcgttgtaccacacctgatttatgcggtagccttctaactactcttctgccttccactcttgccagccttcagtctattttgcacatcaggacttggcagactttttatgtcaagggccagatggtaaaaaatttaggttttgtggcctgcacggtctctgtcatagctgctagaccggagttggggcacaaaagcagccatagagaatatgtaattgaatgaatgtggttgcattccaatacagctttacttataaaaacaggcagtgggctgtggtctgccagcccctgctctacatggcaaccagactacccagtgccttccggtgacttaatctttatagtgttctctaaaaccctccatgatgtgcgtccccaaccccttacctgtctgacttcatcttacgctgttgtctgctttcctccctcctctccagtcacactgaccctctttttcctcagatatgccaggcccattgccacctgattctttgcaccatttattccttctgcctggaacattcatttcttcttaaatgtcaacttctcagtcaggcctacctgaccaaccaccctataaataattacattctagcattatattccctattgcgtgtttttttttttcttcgtaatgcttgtcttctgccatttcttatattatgctatatattgacttaattttttctctttcttttcctgctagaataaatgttccctgaaggcagtaactttgtatcttttctattaagtagtgtatccccaagatctagaacagtgcctgacgtatagtaggtgctcagaaaatatttgttgaatgaacacatagtgtatatttttagggtttaattttttttaaatgagttctttctctctcgccagcaccatatcctttctgtttcacactcttactgttgctctaagctgccctgtcctttccattctcactcccctttattctgactctcatctattagcagtgtgtccataaccttttatttcgtggcatctgggccatcctctacaagtagattgagtgatgtttgtaaaatggaaaaatgattttattattcaaaactctttatttttgattttttgccaattacaatgaagtgcttgtttaaagtacaggatctcttatgaaggaataattgtcaagaaaaaaaagtacgggatctcaggcctcatgccatacctgataagtcataatttcaagcagagggcataagaatcatcataatttaggaagtaacccaagcgattcttactgaacacagttggccaacactggcctgtaaggtaaagttccccagtgagtcagtgagagcctttttgagctgcccctgcctgtctgtcctgccccatccctattgctccacactgcacttcatcttccaacagtttttctatttgcatttctctcaaggcaccatattgattctgtttttgcaaatattcttttccctgtacagaacagaatcttctactctggaaaacttctatttttgtctttcaaaattctgcacgtttgtcactttctctataaagtcttttctgatccccattcccgtgacaaagttaattatgtctttgctgtctgggtactttgtgtaagtaagactctgttctggtgtttattacactgtactgtgtgcctttgcataactgtttgcctgttgtgacctgacctgagccagtagttgtttacgggtattaggtttttattgagaggtactgctgttctttggaggctacagttaaatgagtgttacaaatctcttcccccagaaccgagcttctaaatggcatgttactgtgaaagcaaaactcacactgagttctgccacacacaaaaaaaatttattaggaagcaagcacaaaaatactgttttattaggtggataaaattcctactctctatgcttttagaacatctgacaagcacctttttttttttttttttttttttttttttgagacagagtttcgctctgttgcccaggctggaatgcagtggtgcgatgtcggctcactgcaccctccacctcccaggttcaagcgattctcctgcctcagcctcccgagtagctgggattacaggcgtgtgccaccccacctgggtgatttttgtatttttagtaaagatggggtttcaccatgttggccaggctggtctcaaacttctgacctcagatgatccacctgcctcggcctcccaaggtgctgggattaccggcttgagccaccatgcccggcctgccaagcaccttttatggaaggtttatttaaggcaaaaatgctagtgtgcttttaggattcagctgaacctaattggaatcaaataacagaattcctttatggggaactaacagaaccagcaggagtcataaggctcagggtacagtccctaatagtaggccattagattaactagattcatgtatatgttcatgtaagagcagtggtccctatacagtgtgtagccataacacctttcttgagtaccattcttctctaatactgtcataatttctaatatccactatgttaattactaaattctctggctttgagatgagataaggcttcttgagttgtctcactggagtagggtttttgattacaagctttcctcatttactgatgattatttatatccagtcttgggcttgcatgttaactaagtgctccaggatgaatgtcaggaacctctcactcatgtcctctagtccattaaaacaaataataattacactgtttgtgccaaaataacttaaggaatttaaatcgtaactcttctagaccctggaattgaaacagatgttttacttttaatctctgtatcctcatgcctagtgcagtacctgctcagtgagcctcaagaagtgccagaatgtatgtttgtaatgtcaagcagctaagtgttttgttctgtttgtatttttgtttttaatgccgaattgcagactcttccaacgaattatattaataatatagcaagtttgccatagctgtcagaatatctgtcttaacagtgatacttccttgcatgggctagaatgaggttgtataattccctgggtggggatcaagctgagacctgctcaggatccccagtctccttgtggagagaggttgctctgtcttcctcatgaagtttgcagtgggagtggtgtgtgtgatataaatacagagccctgcttgggaatctaagaaagagttgtgcagtgctggtaagattatagctgaggttaacaaattgtagatctctgttctgacagcagagctagacagctcaaaatataatgtgtttcaccagatgaatttcttatttgtaaagcataactttatcaattcttcactaaaagggaaatgaaagctcagagagtactaaggcatcatgacatatctctagattatactgtcgggagcatcatacttaagttgatttttaagtgtatatttaagtttttttaatcatgggctagaattagagcctaataaggaaagtagtttggtgttagattattaaacaattcataattcttgggccacttggagagtgatttggatcaatgctatttcttcttcaataatctttcattgtttttctgtatttttcatatttctatgaacatgggaagaatcttggatttttaaaaactttacattgataattaatataattatattctgccttcctgtaccgataataaaaactttgtattaacatactcttccacacaagcagagtaaaaaggttgaaattgttgttgtgtcatatgacagcggttaacaaccaaagagctcttaaaaatcagtagggggccaggtgtggtggctcacacctgtaatcccagtactttgtgaggccagagtgggaggatcacttgagaccagaagttcaagaccagcctgaacaacgtagtgagagcccatctctagaaaacattaaaaaattagccaggtgtggtagcgtgtacctgtactcccagctacttgggaggattgcttgatcccagatgttcaaagttgcagtgaaccatgatcacgccactgcactccagcctgggtgacagagtgagaccctgtctctaaaataataataataactcagtagggaaaagattaacccagtagaaaagtggataaaggatatgagcaagcagttgaagaattaaatggccaataaaatatgaaaaatgctgacttaaaaatataagatgtaattaaaacattagtaagataaatgttacctatcaaattgagctgttttcaaagattaaaacttactatgctgggttggcaaatggttaaaaagatgtgtgcactcatacaaagccagtaggaatataagctcatgcaataattctggagaacatttcagcaatcagaaagctttaagacctaataatttcacttatagtaatttaattttaggaaaatatcaaggatttacaaagattaagctccaaaactattgttgatcacaatattattataatagcatttttctaatttttaaggaaactatatgcccttatatgtctttttaaaatatatatatacctatcatttacacttagaaatgaattagtcaaaaaattagtcagaaatcatttataataagtcagcttttgaccactgagttgtttactgtgatcaagaaagcagcaaggcttaaggagttcttattcttttttttttttttttttttgagacggagtctcgctctgtcgcccaggcttgagtgcagtggcgcgatctccgctcactgcaagctccgcctcccgggttcacgccattcaaggagttcttattctaaatgactttatttcatgacaatctgttctctgttctcttctctctgtactcccttggcaatctgtggcaggcacacggcttcagctatcccatgcgaagatggctcctaaatttacatttccagtcatgacttttttttggagttccacacaagtttccaactgccctttgaacatccccacctggatggccttccagcatgacctgttttcttccgtatttgaactagatttttgtagacccccaaggtagtgatagtgatgtgtatgtgtgctttggaagtgggcagggaagagacttaaaaagatttaactgtagaactgaaaaataccatgtgcagtacagcaggttcttgaataacattattctgttcagtgtcgttttgttataatgttgatgagggataaagaatcaattcctggccgaggccaatgtctgtgtggagcatgcaagttctccccatgtctgcgaagttttcttcaggtgctccagtttcctcccacatcccaatgtgtgcatctttgggatgtggactgacgtatctgttatcttagtgtgagtgaatatgggtgtgtgtgagtgcaccttgcagtagaatggcgtcctgcccaaggctggctcccatctcgtgctttgagctcccaggataggccccagccacctgcaaccctaaaactggactaatggcataaataattaccttgtttttattaatctttcttaaatgtgtatatagctcacatttatttcaaggtttaccattagaagtgttttggtctttatttagaagtttggcaatgttttatgaccacaaatatgctgtagaaacttaagttggtttcattatacattgtttcacataaagtttccaagaacctatcgaggctattagtgaggacttactgtacatcttgacatgggagaattgggcattaggcatgagcatgtgccactgcagcaggtggtgggccgcttacgagtctggattaccaatttcatctgaactcttgggtggactatatacatgccttactggagcaaggaattgaagaagaatacagaaggaagttgtttttctctgcctcagttcatatcctgcctctgccatttagagtctctgaccttcggcaatttttttcttttcactccagtttagttttctggtctctaaaatggggggagaaattgtgcctacctaaggtttgctgtaaagaatgaccgaggtaatccatgcctggcctataagtgattagtaaatgttagctgtcagcctctacaaaaggcatagtgctatgtcaaagcatagtgctttgataatatagtttaataggataaattttcagatagtaaaccttttcccttcattcagggtaaaaataatagttctgtaacccatcaaagtttagttctctaagctttgtatccctgttttaaaataattcagctcaaaaaagaaaaataagtttcctggttcagcttcatattgaagccttctaatgctagaaataaatgtctctagattcctcagaggcattgcggacctcttccttccttttattctcagagtgaaaaacaaatttatgctgtttcctatcttaagaacattctcaatcagccaggcacggtgtctcacgcctgtagtcctagcactttgggaggccgaggcaggcagattgcttgagcccaggaggggaagaccagcgtgggcaacgtcgtgagaccttgtctctgaaaacgtaaaaaagaacatccttaatcatatatattttcatatcagaaaagaaaaaaatagtattgttttgttttgtttatgtataaataggtttttctcgatgggaaattgaaacatttaaagttggtgtctctataaggtggcaaattaggaatttacctcagaattatatagtggtggattgcattgtttctttttcaggctttctggtttatagggttatgttggtatttttcgtgttattctttttacaattataatcatgaactgctgcctttcctggggctggtattggcaagttaattgggttggcagatccatgcaggccctgtggagcactcatcccctctcccttcttcctttctcctcatttttcagtattcctgtcaccagagcagagcacaagggacctagggaatgccagataagactaaagtggtaatggccacttttaaataatacatttagatggtgataattgagctatactgggaattatagaacttaacacaattacattaaatttattaaaacttttcttcaaaacgtgagatttaaaacttttctttactctgccttttattttttcaaagaatcctcacttggaattcaaatactgtataggaaacagctaagcactgcctctagtttaaggttggggttaaaacctgcttgtttggcctcttctcatatcacttcattcccaattagtcccctacccctagatacctccctagggctccagggctctaaggaaagagtttagaagtgaataatacaaacagatccccggttgggggataatcctggtgccctcagttttgttctattctgggatgtctggctcattttgacagttatcgataaactcctctcctacccgatgcttgtaattaaaactgctctttcacctagtgtttccattttcatgtacagtgactccattcatttacttacctaggctgaaaaccccgtagtcattcttgactcttcttactctccactcattagcaaatcatgttggttttctgccttcagaatacaccccactttgctactctgggctcagcctctttggtttcttgcccggaaactttaatagctaacttagtcttctcttttcactctttgcttcctgcagtctagtacagtggttaattccatgggttctggaaccagattccctgtcatgtgactttgcgaatactaattagcctttctaaacctcagtttagaggaatgaggatgagaacagtgatactgtttacctcacatggttgtgtcagacaacttgttatgcacaaattacttagataggacatggcaaacagtaaatgtcccataaatgttaggtggtaagagatattgaggttgttgtggttgtttttattcatacagacttgaaactgatattctcaaaacattagtcagatacattctctgttggcttcctggcccttcttagcacaatatccaaaggccttcctgtggcttaccaagccctgcagctctaggttacctggttcctctctgatcacattggcccgtgctcgctgtgttccagccacactggcctgctgctctttcctgccttgttcagcctttgagcactcattttccctctgccacattgctctctcttcagagatttgcccaacttgccgctcccttcatttaagtctaaacccaaatgtcaaccactgttcaagaatatcatgtcatctttctctatatacctaatttttttcatatcttaagaaaatacaacttttcactacctaacattacattatatatctgtttattggctaattatgctgttagaccagatttcatagtacaatagtcccctcttatctgagggcaataccttccgagaccaagtggatgcctgaaactgtggatagtaccaaatcctaatatactatattttttcctgtacatacatacctataataaagcttaatttataaagtaagagattaacaacaacagtaaaataggacaattataacagtatgccagcatcgctactcttgcactttggagctgttattaaataaaataaggtttccttgaacacaagtaccacgacgattgctctgatgacctagaaggctactaaatgactaatgggtggatagcatctacagtgtggatatgctggacaaagggatgattcatcccaggcaaaacggggcaggactgcaaggtatttcagtgcactactcagaatggtgtgcaatttataaactgtttatttctggaattttctattaaattttttggactaagttgaccaaaggtaactgaaatcacagaaagcaaaacttcttataaggagggactatgtaaatgagtgcaagggaccttttctctttggttcatcgttttagccccagtgcctttcatatacatagtcgatgttcaagaggtatttgttaaatggatatatgggatgtgatggggtgagtgactgacaaatattttccccttccttctttacacaagagatgctgaagttaccagatagttggaaaggcccaaattaacttttttgagatgtagagttcaggtttcccccttctttcccatgaataacaatatattgtgtaattcatcataattacttcagttaaccacaaatggagtaattgcattgttaaattagtaccctcttacagttactattcaaggtaaacttctttcctgggtagctagtgacaagaaaatcatttgggctcatcatttattgagcacttaaacccactaagcccatgccacactcctggttgttaccaacctccagtgctgaaagaaggaacttgttaagtctgtctggaatcaggaaaaacagagacatatataatgttctccaaaaattctgctgcttataaagagcagtgaattcagagtgtctagggaaacaggatttttaaataaggatttaggtttaagatgcctcttagtttcttagggatgggaagtgagttttaaaaattagcatttttcttttaatatgatagtagaacaattcggttactgtttgataactttatactaattcattgactttattatatgaagaaataaaggacttcaagaatagctcataacaaaccgggtggcgtggtggctcaagcctgtaattccagcactttgggaggctgaggcaggaggatcacttgaggtcaggagtttaagaccagcttggccaacatggtgaaaccctgtatctactaaaaatacaaaaattagccgggcctggcagcatgcacctgtaatcccagctactcgggaggccgaggcaggagaatcgcttgaacctggaaggtggaggcagtgagctgagatcgtgccactgcactctagcctggtcaacagagcaagactctgtaaaaaaacaaacaaaaacaagaatagctcataaccttgaacgtctttgtttgttttcttcataattcctgagaattttttaaaaaataccattaccaatttcagttaattatcatgtacacctagaatatggaagttattttgtgctttttattttgggcctcttttctacctttaagcttaacatccaacagtcttctgaaccatatcttgttataatctgagcaaataattttctttctgattgaaagagggtaaacataagctaatttgtttagtcctttttttaaaaaaaaatcactgacactaaatgagttaatgtttggagtttagttgacctcctaaattatccctattacaacatagtaagacaccatatctacaaaaaatacaaaaattaaccagcctggtgacacgtgcttatagtcctagctacttaggaggctgaggtgggagaatcacctgagcccaggaggttgaggccgcagtgagccatgattgcatcactgcactcgaacctgagcaacagagccagaccctgtctcaaaaaaaaaaaaaattggtaccattcagagacagtgtcagatggggtgttgactgggttggttcacctatcagatggtaaagtaggtgttctgaggtgagctcaaggaagattgaaatctcccatggagcaggacaaaaacgtacttgatcttgattatctgtatttataaagattgtcaaagtaggaccaagaaatttttctgatctttggagttatcactgtgaagcagaattcaccagtccttgttcatcaacctatcttgtgttccctataaatgttaatgtagaatagccttatcttatctacaaaatattatttttattagaagaatctaagtgtaaatcagtagccaaaaatcagagatctgatttattagctgttctctctttttatttatacatggactttaataatcattattatcattattacag

SEQ ID NO: 5 - DNA, 20 bp, ARTIFICIAL SEQUENCE
  Forward Primer
tattctgtcttcggcagagg

SEQ ID NO: 6 - DNA, 20 bp, ARTIFICIAL SEQUENCE
  Reverse Primer
agcaagcaaagaaaggcgag

SEQ ID NO: 7 - DNA, 23 bp, ARTIFICIAL SEQUENCE
  SNaPshot Primer
aggacttttattattgcttgtta

SEQ ID NO: 8 - DNA, 20 bp, ARTIFICIAL SEQUENCE
  Forward Primer
attgcttgggaggttgttgg

SEQ ID NO: 9 - DNA, 20 bp, ARTIFICIAL SEQUENCE
  Reverse Primer
tttcactggagttgtgggag

SEQ ID NO: 10 - DNA, 17 bp, ARTIFICIAL SEQUENCE
  SNaPshot Primer
gatctcccagtgactca